(19) United States
(12) Patent Application Publication
(10) Pub. No.: US 2008/0183232 A1
(43) Pub. Date: Jul. 31, 2008

(54) Method and system for determining cardiac function

(76) Inventors: Gregory I. Voss, Solana Beach, CA (US); Bernhard B. Sterling, Danville, CA (US); Anthony J. Bergman, New Port Richey, FL (US); Alexander K. Mills, San Antonio, TX (US); Donna Wall, San Antonio, TX (US)

Correspondence Address: Francis Law Group, 1942 Embarcadero, Oakland, CA, 94606, US

(21) Appl. No.: 11/700,328

(22) Filed: Jan. 30, 2007

(51) Int. Cl. A61N 1/00

(52) U.S. Cl. 607/24; 607/17

(57) ABSTRACT

A method for determining a physiologic characteristic associated with cardiac function in a subject comprising the steps of providing at least one electromagnetic radiation absorption measurement, providing demographic information reflecting the subject's physical condition, determining a temporal plethysmographic value from the electromagnetic radiation absorption measurement, and determining at least one physiologic characteristic from the temporal plethysmographic value and demographic information by using a predetermined phenomenological model that is adapted to provide an estimate of a blood volume-time relationship proximate the heart and compute at least one physiologic characteristic associated with cardiac function based on the estimated blood volume-time relationship.

### FIELD OF THE PRESENT INVENTION

The present invention relates generally to the field of signal processing and analysis. More specifically, the invention relates to a method and system for determining physiological characteristics associated with cardiac function.

### BACKGROUND OF THE INVENTION

The study of the performance and properties of the physiology (including notably the cardiovascular system) of a living subject has proven useful for diagnosing and assessing any number of conditions or diseases within the subject. The performance of the cardiovascular system, including the heart, has characteristically been measured in terms of several different parameters, including the stroke volume and cardiac output of the heart.

Noninvasive estimates of cardiac output (“CO”) can be obtained using the well known technique of impedance cardiography (“ICG”). Strictly speaking, impedance cardiography, also known as thoracic bioimpedance or impedance plethysmography, is used to measure the stroke volume (SV) of the heart. As shown in the following equation, when the stroke volume is multiplied by heart rate, cardiac output can be obtained.

CO=SV=heart rate

During impedance cardiography, a constant alternating current, with a frequency typically in the range of approx. 65-75 kHz, I(t), is applied across the thorax. The resulting voltage, V(t), is used to calculate impedance. Because the impedance is assumed to be purely resistive, the total impedance, Z_{T}(t), is calculated by Ohm's Law. The total impedance consists generally of a constant base impedance, Zₒ, and time-varying impedance, Z_{c}(t), as shown in the following equation:

Z_{T}(t)=V(t)/I(t)=Z_{c}(t)+Z_{c}(t)

The time-varying impedance is believed to reflect the change in blood resistivity as it transverses through the aorta.

Stroke volume is typically calculated from one of three well known equations, based on this impedance change:

SV=p(L²/Z²₀)*(LVET(dZ(t)/dtₘₐₓ)) (SV₁)

SV=(L³/4.25Zₒ)*(LVET(dZ(t)/dtₘₐₓ)) (SV₂)

SV=(δ((0.17H)³/4.25Zₒ))*(LVET(dZ(t)/dtₘₐₓ)) (SV₃)

where:
L=distance between the inner electrodes in cm;
LVET=ventricular ejection time in seconds;
Z₀=base impedance in ohms;
dZ(t)/dtₘₐₓ=magnitude of the largest negative derivative of the impedance change, Z_{c}(t), occurring during systole in ohms/s;
p=resistivity of blood in ohms/cm;
H=subject height in cm; and
δ=weight correction factor.

Two key parameters present in Eqns. SV₁, SV₂ and SV₃ above are dZ(t)/dtₘₐₓ and LVET.

These parameters are found from features commonly referred to as fiducial points, which are present in the inverted first derivative of the impedance waveform, i.e. dZ(t)/dtₘₐₓ.

As described by Lababidi, Z., et al, “The first derivative thoracic impedance cardiogram”, Circulation, vol. 41, pp. 651-658 (1970), the value of dZ(t)/dtₘₐₓ is generally determined from the time at which the inverted derivative value has the highest amplitude, also commonly referred to as the “C point”. The value of dZ(t)/dtₘₐₓ is typically calculated as this amplitude value.

LVET corresponds generally to the time during which the aortic valve is open. The point in time associated with aortic valve opening, also commonly known as the “B point”, is generally determined as the time associated with the onset of the rapid upstroke (a slight inflection) in dZ(t)/dtₘₐₓ before the occurrence of C point.

The time associated with aortic valve closing, also known as the “X point”, is generally determined as the time associated with the inverted derivative global minimum, which occurs after the C point.

In addition to the foregoing “B”, “C”, and “X” points, the so-called “O point” has also found utility in the analysis of the cardiac muscle. The O point represents the time of opening of the mitral valve of the heart. The O point is generally determined as the time associated with the first peak after the X point. The time difference between aortic valve closing and mitral valve opening is known as the isovolumetric relaxation time (“IVRT”). However, to date, the O point has not found substantial utility in the stroke volume calculation.

Impedance cardiography further requires recording of the subject's electrocardiogram (“ECG”) in conjunction with the thoracic impedance waveform previously described. Processing of the impedance waveform for hemodynamic analysis generally requires the use of ECG fiducial points as landmarks. Processing of the impedance waveform is generally performed on a beat-by-beat basis, with the ECG being used for beat detection.

In addition, detection of some fiducial points of the impedance signal may require the use of ECG fiducial points as landmarks. Specifically, individual beats are identified by detecting the presence of QRS complexes within the ECG. The peak of the r-wave (commonly referred to as the “R point”) in the QRS complex is also detected, as well as the onset of depolarization of the QRS complex (“Q point”).

Under the prior art approaches, the aforementioned beats are scrutinized for artifacts (due to motion of the subject, or other such causes), through comparatively simple rules, such as the evaluation of calculated parameter values outside a typical numeric range. Illustrative is the well-known “Weissler window” disclosed in Weissler, et al., “Relationships Between Left Ventricular Ejection Time, Stroke Volume, and Heart Rate In Normal Individuals and Patients With Cardiovascular Disease”, Am. Heart J., vol. 62, pp. 367-78 (1961) which defines the X point search interval based upon the heart rate and gender of a given individual.

The Weissler regression equation is based on 121 normal males and 90 normal females. Although the relationship between heart rate and LVET is linear for normal individuals, in another work Weissler et al. found that this relationship does not hold for abnormal patients. In 12 non-valvular CHF patients with COs ranging from 2.1-5.8 L/min, 9 patients had a significant decrease (p<0.05) in ejection time relative to heart rate. See Weissler, et al., “Systolic Time Intervals in Heart Failure in Man”, Circulation, vol. 37, pp. 149-59 (1968). Thus, when applying such criteria, the true X points in CHF or other cardiovascular patients may be erroneously rejected because these X points lie outside of the Weissler window.

Other such “parametric” rejection rules can include, for example, (i) LVET outside of a desired range, (ii) detection of a pacing spike with the left/right values of ΔZ(t) (also referred to as Delta Z), (iii) d²Z/dt²_{MAX}=0, and (iv) dZ/dt_{MAX}=0 (or less than a percentage of the median value of the most recent beats).

Parameter values from the beginning beats (i.e. those not rejected by the aforementioned parametric criteria) are then typically averaged as a mean, based on a beat average number chosen by the user.

Aside from erroneous rejection of beats, as described above in the context of the Weissler window, other problems with prior art heart rate (or beat) analysis and rejection approaches exist. Specifically, significant instabilities in various of the monitored or derived parameters, such as ECG and left/right ΔZ(t), can result. Such instabilities can reduce both the accuracy and clinical robustness of the measurement process. Erroneous pacing spike detection can also occur during a time interval that does not overlap with a valid B, C, or X point. Additionally, when the electrodes are disconnected, the “flat-line” ECG and Delta Z signals may provide a non-zero cardiac output (CO) estimate.

Still another distinct deficiency with the prior art analysis and rejection schemes relates to their lack of discrimination between different types of subjects. This lack of discrimination has two primary outgrowths, which (i) cause the system to simply not function due to not being able to measure one or more necessary parameters and (ii) imbue the user or operator with somewhat of a false sense of security that all types of subjects (regardless of their peculiar waveforms, arrhythmias or defects) could be successfully monitored, including generating highly suspect or even erroneous data without otherwise alerting the user as to the potential for degraded accuracy. Without any sort of contraindication (or even metric advising on the confidence level of the data or results), the user/operator has no way of knowing, other than perhaps via innate experience or knowledge, whether any given data is valid or accurate.

It is thus apparent that what is needed are improved methods and apparatus for assessing physiologic parameters of a living subject; particularly, physiologic parameters associated with cardiac function. Such methods and apparatus would ideally be completely non-invasive, accurate, easily adapted to the varying physiology of different subjects, and would produce reliable and stable results under a variety of different operating conditions. These methods and apparatus would be particularly adapted to processing optimum signals and waveforms, and would allow for monitoring of a broader range of patient types and conditions.

### SUMMARY OF THE INVENTION

In accordance with the above objects and those that will be mentioned and will become apparent below, in one embodiment of the invention there is provided a method of determining a physiologic characteristic associated with cardiac function in a subject, comprising the steps of: (i) providing at least one non-invasive measurement representing a physiological parameter of the subject, the non-invasive measurement comprising electromagnetic radiation absorption; (ii) determining at least one temporal plethysmographic value from the electromagnetic radiation absorption; (iii) providing demographic information, the demographic information including information reflecting the subject's physical condition (e.g., age, height, weight); and (iv) determining at least one physiologic characteristic from the temporal plethysmographic value and demographic information by using a predetermined phenomenological model, the model being adapted to provide an estimate of a blood volume-time relationship proximate the heart from non-pressure related measurements and compute at least one physiologic characteristic associated with cardiac function based on the estimated blood volume-time relationship.

In another embodiment of the invention, the method of determining a physiologic characteristic associated with cardiac function in a subject comprises the steps of: (i) providing at least first and second non-invasive measurements representing physiological parameters of the subject, the first non-invasive measurement comprising an electrical measurement of the heart, the second non-invasive measurement comprising electromagnetic radiation absorption; (ii) determining at least one temporal plethysmographic value from the electromagnetic radiation absorption; (iii) determining at least one temporal relationship from the electrical measurement of the heart, (iv) providing demographic information, the demographic information including information reflecting the subject's physical condition (e.g., age, height, weight); and (v) determining at least one physiologic characteristic from the temporal relationship, temporal plethysmographic value and demographic information by using a predetermined phenomenological model, the model being adapted to provide an estimate of a blood volume-time relationship proximate the heart from non-pressure related measurements and compute at least one physiologic characteristic associated with cardiac function based on the estimated blood volume-time relationship.

In another embodiment of the invention, the method of determining a physiologic characteristic associated with cardiac function in a subject comprises the steps of: (i) providing at least one non-invasive measurement representing a physiological parameter of the subject, the non-invasive measurement comprising electromagnetic radiation absorption; (ii) determining at least one temporal plethysmographic value from the electromagnetic radiation absorption, (iii) performing non-linear scaling of the temporal plethysmographic value according to a physiological parameter selected from the group consisting of minimum blood pressure, maximum blood pressure and a predetermined blood pressure, the predetermined blood pressure being greater than the minimum blood pressure and less than the maximum blood pressure; and (iv) determining a time-varied blood pressure from the scaled temporal plethysmographic value.

In yet another embodiment of the invention, the method of determining a physiologic characteristic associated with cardiac function in a subject comprises the steps of: (i) providing at least first and second non-invasive measurements representing physiological parameters of the subject, the first non-invasive measurement comprising an electrical measurement of the heart, the second non-invasive measurement comprising electromagnetic radiation absorption; (ii) providing demographic information, the demographic information including information reflecting the subject's physical condition (e.g., age, height, weight); (iii) determining temporal arterial pressure from the first and second non-invasive measurements and the demographic information; (iv) determining the extent of a pressure pulse from the first and second non-invasive measurements; (v) determining heart rate from the first and second non-invasive measurements; (vi) determining stroke volume from the temporal arterial pressure, extent of a pressure pulse and demographic information; and (vii) determining cardiac output from the stroke volume and the heart rate.

In accordance with another embodiment of the invention, there is provided a system for determining a physiologic characteristic associated with cardiac function in a subject, comprising: (i) interface means adapted to receive at least first and second non-invasive measurements representing physiological parameters of the subject, the first non-invasive measurement comprising an electrical measurement of the heart and the second non-invasive measurement comprising electromagnetic radiation absorption, and demographic information, the demographic information including information reflecting the subject's physical condition; (ii) means for determining at least one temporal relationship from the electrical measurement of the heart; (iii) means for determining at least one temporal plethysmographic value from the electromagnetic radiation absorption; and (iv) means for determining at least one physiologic characteristic from the temporal relationship, temporal plethysmographic value and demographic information by using a predetermined phenomenological model, the model being adapted to provide an estimate of a blood volume-time relationship proximate the heart and compute at least one physiologic characteristic associated with cardiac function based on the estimated blood volume-time relationship.

In another embodiment, the system for determining a physiologic characteristic associated with cardiac function in a subject comprises: (i) interface means adapted to receive at least one non-invasive measurement representing a physiological parameter of the subject, said non-invasive measurement comprising electromagnetic radiation absorption, (ii) means for determining at least one temporal plethysmographic value from said electromagnetic radiation absorption, (iii) means for performing non-linear scaling of said temporal plethysmographic value according to a physiological parameter selected from the group consisting of minimum blood pressure, maximum blood pressure and a predetermined blood pressure, said predetermined blood pressure being greater than said minimum blood pressure and less than said maximum blood pressure, and (iv) means for determining a time-varied blood pressure from said scaled temporal plethysmographic value.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of a human heart, showing the pulmonary and systemic circulation sections;

FIG. 2 is a graphical illustration of an R wave portion of an electrocardiogram waveform and the related plethysmographic waveform;

FIG. 3 is one embodiment of a photoplethysmogram system, according to the invention;

FIG. 4 is another embodiment of a photoplethysmogram system, according to the invention;

FIG. 5 is a flow chart illustrating one embodiment of a method for determining a physiological characteristic associated with cardiac function, according to the invention;

FIG. 6 is a schematic illustration of arterial blood pressure over a sequence of heart beats;

FIG. 7 is a schematic illustration of an optical photoplethysmograph (“OP”) signal and an ECG signal that correspond to the arterial pressure trace shown in FIG. 6, according to the invention;

FIG. 8 is a flow chart illustrating one embodiment of an arterial pressure-based method for determining a physiological characteristic associated with cardiac function, according to the invention;

FIG. 9 is a flow chart illustrating another embodiment of a method for determining a physiological characteristic associated with cardiac function, according to the invention;

FIG. 10 is a graphical illustration of a demographic correlation based on weight and height, according to the invention;

FIGS. 11A-11E are graphical illustrations showing the relationships between arterial pressure, aortic cross-sectional area and arterial compliance for various weights and heights, according to the invention;

FIG. 12 is a graphical illustration of a blood flow acceleration duration score as a function of a plurality of contiguous samples, wherein the acceleration is positive, according to the invention; and

FIG. 13 is a graphical illustration of a blood flow ratio score as a function of the ratio of (test point volume−diastolic volume):(peak systolic volume−diastolic volume), according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “signal”, as used herein, is meant to mean and include, without limitation, an analog electrical waveform or a digital representation thereof, which is collected from a biological or physiological sensor and/or transmitted by an apparatus or system of the invention.

The term “heart rate”, as used herein, is meant to mean and include, without limitation, a measure of cardiac activity. As is well known in the art, “heart rate” is typically expressed as the number of beats per minute.

The term “stroke volume”, as used herein, is meant to mean and include, without limitation, a measure of volume pumped per beat, which is typically expressed as the volume of blood pumped from a ventricle of the heart in one beat.

The terms “cardiac output” and “minute volume”, as used herein, are meant to mean and include, without limitation, a measure of the volume pumped per time, which is typically expressed as the volume of blood ejected from the left side of the heart in one minute, in units of liters per minute (1/min).

The term “cardiac index”, as used herein, is meant to mean and include, without limitation, a cardiodynamic measure based on the cardiac output. Cardiac index is typically expressed as the amount of blood the left ventricle ejects into the systemic circulation in one minute, divided by the body surface area (“BSA”), i.e. the total surface area of the human body. The cardiac index typically has units of (1/min)/m².

The term “pulse transit time”, as used herein, is meant to mean the time required to transit blood from the left ventricle to an accessible measurement site of the arterial pulse wave. In one embodiment of the invention, “pulse transit time” means the time period between the QRS complex of the ECG and the beginning of the systolic upstroke of an optical volumetric plethysmographic measurement.

The terms “patient” and “subject”, as used herein, is meant to mean and include humans and animals.

The present invention substantially reduces or eliminates the disadvantages and drawbacks associated with conventional signal processing systems, apparatus and techniques. As discussed in detail below, the invention(s) includes embodiments for determining at least one physiologic characteristic that is associated with cardiac function (also referred to hereinafter as a cardiodynamic function). In one embodiment of the invention, the system includes a pulse oximetry or optical plethysmography type tissue probe having an electromagnetic radiation emitter and a detector configured to receive the radiation after absorbance through the patient tissue, and a controller for determining one or more physiologic characteristics based on the absorbance.

According to the invention, the probes are adapted for use with (or on) arms, hands, fingers, feet, toes, inner ears (or ear canals), earlobes, nares, lips, tongue, and the like, or on the back, neck or forehead. The probes can also be disposed on one or multiple sites; preferably, at a skin site where there is sufficient positive arterial blood flow access and which are substantially supplied by arterial blood during periods of peripheral vasoconstriction.

Preferably, the probe or probes are disposed at a location on the body that reflects, as accurately as possible, the pressure waveform inside the aorta. Two optimum locations are thus the forehead (over the eyebrow) and the side of the neck. These are excellent sites, since they are proximate thin major vessels that branch off the central circulatory system and, hence, the pressure waveform would be substantially unaltered.

According to the invention, the radiation emitters of the invention can utilize a single wavelength or a plurality of discrete wavelengths. The emitters can also be adapted to provide visible light, infrared light, e.g. near- and/or mid-infrared, and ultraviolet light.

In one embodiment, the emitters are adapted to provide near-infrared light. As is known in the art, near-infrared radiation facilitates deeper penetration of tissue. A near-infrared wavelength, e.g., 1200-1250 nm, is particularly preferable at a site on the body where blood has very little absorbance, such as the side of the neck. When employed at such a location, one has a better chance of acquiring a reflective signal that is affected by the positive nature of the target artery.

Further, one is not limited to the use of hemoglobin absorbance measurements to determine a physiologic characteristic, according to the methods of the invention. One can use the increase in water in a vessel as it expands. By way of example, take a digit with a pulse wave going through it. As the pulse wave transitions through the digit, more water is being disposed therein. A measure of the increase in water would thus reflect the transition of the pulse or pressure waveform. This approach would similarly be beneficial for probing deeper tissue regions on the body.

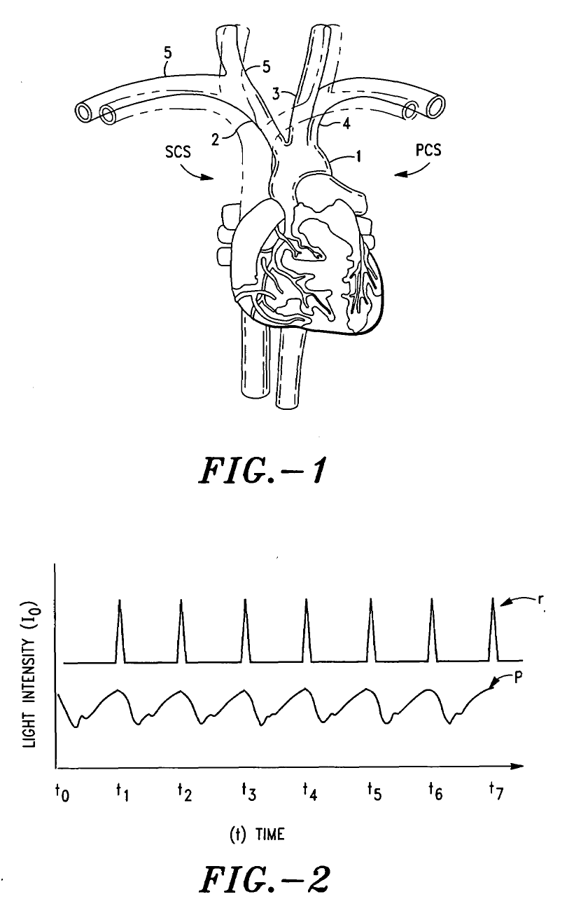

As is well known in the art, functionally, the heart is divided into two sides or sections. Referring to FIG. 1, the right or pulmonary circulation section (designated “PCS”) receives blood from the veins of the body and pumps it through the lungs; the left or systemic circulation section (designated “SCS”) receives the blood from the lungs and pumps it to the body. The blood is then collected in the veins to be returned to the right side of the heart.

As illustrated in FIG. 1, the arterial system begins at the aorta 1, to which the left ventricle of the heart pumps. The aorta 1 passes down (caudad) through the body, providing arterial branches to organs, and terminates as a bifurcation, i.e. creating the iliac arteries.

The first three branches of the aorta 1 are the brachiocephalic or innominate artery 2, the left (common) carotid artery 3, and the left subclavian artery 4. The brachiocephalic artery 2 branches into the right subclavian 5 and right (common) 6 carotid arteries. These arteries provide the blood supply for the head and upper extremities.

The brachiocephalic or innominate artery 2 is the first branch of the aorta 1. The innominate artery 2, in turn, branches into the right subclavian 5 and right carotid arteries 6. In contrast, the left subclavian 4 and left carotid arteries 3 originate directly off the aortic arch. Thus, the subclavian 4 and carotid arteries 3, as well as their branches, have different paths from their counterparts on the opposite side of the body.

The functioning of the heart may be quantified in several ways. These measures of heart function are referred to herein, without limitation, as measures of physiologic characteristics associated with cardiac function (or, alternatively, cardiodynamic function). According to the invention, the physiological characteristics that are associated with cardiac function include, without limitation, heart rate, pulse transit time, wave velocity, central and peripheral blood flow, perfusion, contractility and vasoconstriction. Cardiac functions include, without limitation, stroke volume, cardiac output and cardiac index.

A direct measurement of most cardiodynamic functions requires invasive, or at least highly detailed measurements, of the function of the heart. For example, stroke volume (“SV”), i.e. the volume of blood ejected from left ventricle per beat, is a function or characteristic that is very difficult to measure directly in a patient.

Typically, cardiodynamic functions are measured using indirect measurements and data, including, but not limited to, in vivo or external blood pressure measurements and ECG measurements, which are coupled with demographic information and analyzed using either a phenomenological model or correlation. Alternatively, a system may determine one or more cardiodynamic functions based on indirect measurements and data using physiologically based or self-taught neural, net-like methods and data obtained using an independent measure of cardiodynamic function.

As discussed in detail below, embodiments are presented herein for estimating physiologic characteristics that are associated with cardiac function from the analysis of one or more measurements on a body. In one embodiment, one or more of the measurements comprise noninvasive measurements, which can include, but are not limited to, electrical measurements of the heart, electromagnetic radiation absorption measurements through tissue, and/or the determination of body-part size changes as the result of blood flow (i.e. plethysmographic measurements).

According to the invention, the electrical measurements include, but are not limited to, ECG measurements. Electromagnetic radiation absorption measurements include, but are not limited to, measurements of the absorption of light through the body, where the term “light” refers, without limitation, to electromagnetic radiation in the infrared or visible regions. Plethysmographic measurements include, but are not limited to, electromagnetic radiation absorption measurements through portions of the body having measurable blood flow. The cardiac functions can include, but are not limited to, heart rate, stroke volume, cardiac output and cardiac index.

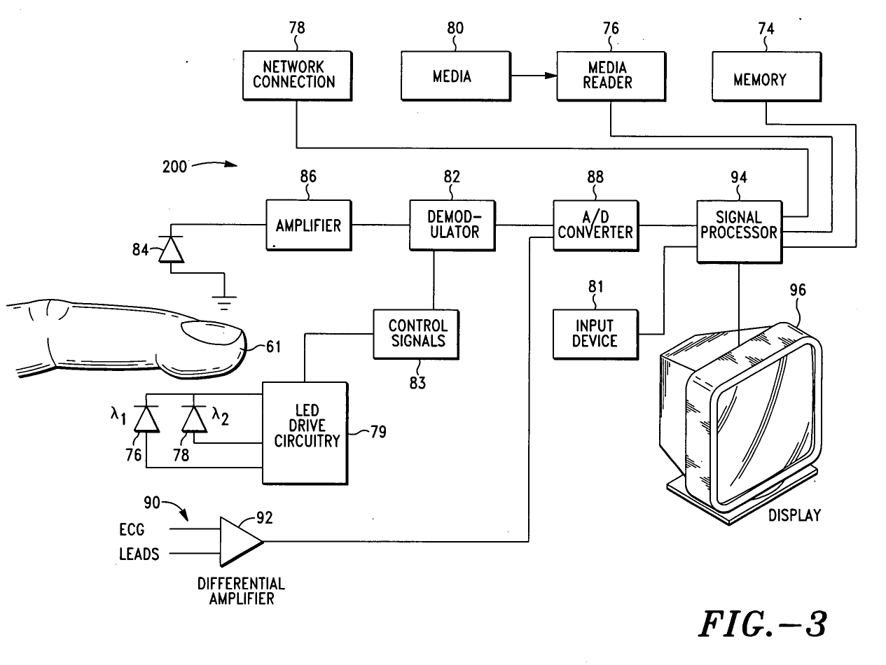

In one embodiment of the invention, an electrical measurement comprises the electric potential of the heart, as measured by an electrocardiogram (ECG). Referring now to FIG. 2, there is shown a graphical illustration of an “R wave” portion of an ECG waveform (designated “r”) and the related plethysmographic waveform (designated “p”). As will be appreciated by one having ordinary skill in the art, the ECG waveform comprises a complex waveform having several components that correspond to electrical heart activity. The QRS component relates to ventricular heart contraction.

The R wave portion of the QRS component is typically the steepest wave therein, having the largest amplitude and slope, and can be used for indicating the onset of cardiovascular activity. The arterial pulsed blood pulse flows mechanically and its appearance in any part of the body typically follows the R wave of the electrical heart activity by a determinable period of time that remains essentially constant for a given patient. See, e.g., Goodlin et al., Systolic Time Intervals in the Fetus and Neonate, Obstetrics and Gynecology, vol. 39, No. 2 (February 1972) and U.S. Pat. No. 3,734,086.

According to the invention, the ECG leads are disposed on the body or torso at a location that facilitates determination of the onset of the QRS complex. In one embodiment, two-lead ECG electrodes are disposed on the torso.

In another embodiment, a measurement of the blood flow through the body is obtained using a photoplethysmographic tissue probe. In one embodiment, the photoplethysmographic tissue probe is configured to communicate with or accept a body part, such as a finger, whereby one or more electromagnetic radiation emitters are disposed on one side of the tissue opposite one or more detectors to accept radiation from the emitters after passing through the tissue.

An example of a photoplethysmographic tissue probe is, for example, a pulse oximeter. Examples of pulse oximeters (or optical probes) are set forth in U.S. Pat. No. 6,537,225; which is incorporated by reference herein.

Although pulse oximeters are typically configured to measure oxygen levels in the blood, the temporal output of pulse oximeters provides an indication of the amount of blood flowing through the probed tissue. The term “optical photoplethysmograph” (“OP”) is thus used herein to describe a preferred device for obtaining temporal measurements regarding blood flow through the tissue. It is, however, to be understood that any probe that provides temporal measurements regarding blood flow through the tissue can be used with the methods disclosed herein, regardless of the intended use of the probe. Thus, for example, a pulse oximeter can be employed as an OP probe in the methods described herein.

In various embodiments of the invention, measurements, such as ECG and electromagnetic radiation measurements, are employed to derive a number of cardiodynamic and/or physiological properties. In at least one embodiment, the measurements are transmitted to a controller to facilitate data collection, storage and analysis. In one embodiment, the controller includes a computing device, which can comprise, without limitation, a personal computer (“PC”) with a monitor. As discussed in detail below, the controller includes programming and algorithms for the calculation of variables not measured directly.

In general, the methods described herein provide an estimate of cardiodynamic function from measurements on a patient, and are not limited to a specific device or type of measurement. For illustrative purposes, the methods will be described with reference to specific embodiments of measurement devices that can be employed within the scope of the invention to obtain or determine cardiodynamic functions or other physiologic characteristics. The methods are thus not meant to limit the scope of the invention in any manner.

Referring now to FIG. 3, there is shown one embodiment of a photoplethysmogram system 200 that can be employed to obtain one or more non-invasive measurements for subsequent analysis according to the inventive methods. As illustrated in FIG. 3, the system 200 includes two emitters 76, 78 and detector 84, which are positioned adjacent the tissue being analyzed, i.e. finger 61.

Two lights are emitted by the emitters 76, 78; in one embodiment, a first light having a discrete wavelength in the range of approximately 650-670 nanometers in the red range and a second light having a discrete wavelength in the range of approximately 800-950 nanometers. The lights, in the illustrated embodiment, are transmitted through finger 61 via emitters 76, 78 and detected by detector 84.

As indicated above, in an alternative embodiment, one or more emitters having longer wavelengths, e.g., up to 2500 nm, are employed to enable deeper tissue penetration.

The emitters 76, 78 are driven by drive circuitry 79, which is, in turn, governed by control signal circuitry 83. Detector 84 is in communication with or connected to amplifier 86. The signal from amplifier 86 is transmitted to demodulator 82, which is also synchronized to control signal circuitry 79. The demodulator 82, which is employed in most photoplethysmogram systems, removes any common mode signals present and splits the time multiplexed signal into two (2) channels, one representing the red voltage (or optical) signal and the other representing the infrared voltage (or optical) signal.

The signal from the demodulator 82 is transmitted to an analog-digital converter 88. As will be appreciated by one having skill in the art, the output signal from the demodulator 82 would be a time multiplexed signal comprising (i) a background signal, (ii) the red light range signal and (iii) the infrared light range signal.

The desired computations are performed on the output from the converter 88 by signal processor 94 and the results transmitted to and displayed by display 96.

As indicated, emitters 76, 78 operate (or provide light having) specific wavelengths, such as from 650-670 nm and from 800-950 nm. Thus, for example, in one embodiment, emitter 76 operates at approximately 660 nm and emitter 78 operates at approximately 880 nm. According to the invention, emitters 76, 78 can comprise light emitting diodes (LEDs) or laser diodes.

In an alternative embodiment, the transmitted light is provided with a tunable emitter that alternates between two or more wavelengths. In any case, it is preferable that the light is either continuous or pulses at a rate of no less than 2 kHz to provide adequate resolution of the pulse.

As illustrated in FIG. 3, ECG leads 90 are preferably connected to differential amplifier 92; the signal from the amplifier 92 being sent to an analog-to-digital converter 88. In one embodiment, the output from the converter 88 is processed by signal processor 94. The output from the processor 94 is then transmitted to display 96.

In a preferred embodiment, the signal processor 94 includes or has access to memory 74, and optionally has access to a media reader 76 and network connection 78. According to the invention, the media reader can include, without limitation, a DVD or CD-ROM reader and the network connection 78 can include, without limitation, a wired or wireless Internet connection.

Programming for the signal processor 72 can be provided through media 80 transmitted to media reader 76, or through the network connection 78. In addition, information from signal processor 94, such as raw or processed information from detector 84, can be stored on media 80 or transmitted over network connection 78 to another computer or computer system for storage or processing.

In one embodiment of the invention, the signal processor 94 is adapted to receive (or in some embodiments requires) patient information, such as a patient's name or identification number, as a marker of information. The patient information can also comprise data, such as a patient's gender, age, weight, BSA, or other demographic information, for use in processing a signal.

As illustrated in FIG. 3, in one embodiment of the invention, the system 200 includes an optional input device 81. The input device 81 is preferably in communication with the signal processor 94, which, through display 96 provides prompts for the input of information. Input device 81 can be, for example, a keyboard, a mouse, a joystick, or other type of input device.

Alternatively, patient information can be supplied to the signal processor 94 from another computer or computer system via the network connection 78.

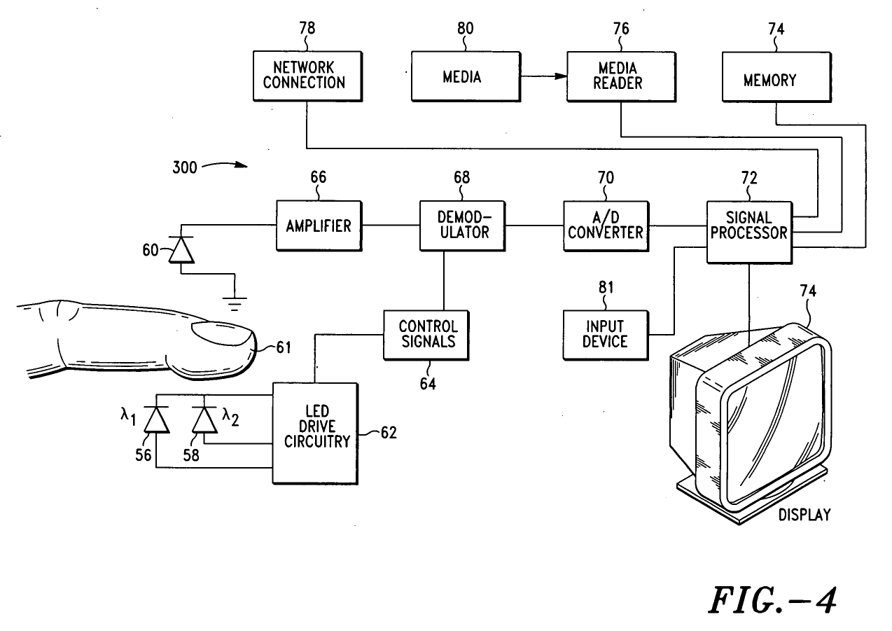

Referring now to FIG. 4, there is shown another embodiment of a photoplethysmogram system 300 that can be employed to obtain one or more noninvasive measurements for subsequent analysis according to the invention. As illustrated in FIG. 4, the system 300 similarly includes two emitters 56, 58 that are connected via LED drive circuitry 62 and control signal circuitry 64 to demodulator 68. The signals from the detector 60 are amplified by amplifier 66 and transmitted to demodulator 68. The output from the demodulator 68 is transmitted to A/D converter 70.

Referring now to FIG. 5, there is shown a flow chart 400 illustrating one embodiment of a method for determining a physiologic characteristic associated with cardiac function, according to the invention. As illustrated in FIG. 5, the method includes the following steps: Obtain Measurements (Block 410); Obtain Demographic Information (Block 420); Analyze Measurement(s) and Demographic Information (Block 430); and Display Physiologic Characteristic(s) (Block 440).

As illustrated in FIG. 5, the output of Block 410 is denoted as measurement(s) M. According to the invention, measurement(s) M from Block 410 can be provided by systems 200 and 300, i.e. electromagnetic radiation measurements, which are shown in FIGS. 2 and 3, respectively. The measurements M can also include, without limitation, electrical measurements of the heart, such as ECG measurements.

In one embodiment of the invention, the measurement(s) M comprise raw signals or amplified signals, such as signals from one or more of amplifiers 66, 86, or 92. In one embodiment, the signals are subjected to further processing, such as the processing of the signal from detector 60 or 84 in demodulator 68 or 82, respectively, wherein pairs of optical transmission measurements through tissue are converted to the temporal plethysmographic measurements. The temporal plethysmographic measurements can be obtained, for example, by analyzing the output of a pulse oximeter.

According to the invention, the signals from detectors 60, 84 can be subjected to additional processing to optimize the signals, e.g., minimize undesirable signals components and/or artifacts. Such processing is set forth in Co-Pending application Ser. Nos. 11/270,240 and 11/270,241, filed Nov. 8, 2005; which are incorporated by reference herein in their entirety.

Optionally, Block 410 can obtain measurements from other diagnostic devices including, without limitation, arterial tonometers, Doppler transducers, pneumo plethysmographs and circumferential strain gauges. Alternatively, under inflated arterial cuffs can be used to obtain blood volume and timing information.

According to the invention, the demographic information obtained in Block 420 can include, without limitation, one or more characteristics directly related to the patient, such as the patient's age, gender, weight, height, body mass index (“BSA”), disease condition(s), therapy, diagnosis, etc. (i.e. patient's physical condition) and other information acquired from publications, third party studies, other patients, etc.

The demographic information can be provided to Block 420, for example, by an input device 81, or the transfer of data over a network connection 78. The output of Block 420 is designated, in general and without limitation, as demographic information D.

As illustrated in FIG. 5, measurement(s) M and demographic information D are provided from Blocks 410 and 420, respectively, as inputs to Block 430. In Block 430 measurement(s) M and demographic information D are analyzed to determine at least one physiologic characteristic C, which is associated with cardiac function.

The methods of Block 430 can be carried out, in general, in hardware, software, analogue circuitry, or some combination thereof. Several embodiments described herein utilize one or more noninvasive measurement(s) M to generate one or more physiologic characteristics C.

According to the invention, the methods of Block 430 include, but are not limited to, models or correlations that are empirical or that are based on phenomenological or detailed models of the circulatory system. In one embodiment, Block 430 determines one or more physiologic characteristics C for each heartbeat. Thus, for example, measurement(s) M are analyzed in Block 430 to identify each heartbeat and analyzes M for each heartbeat. In another embodiment, Block 430 analyzes several heartbeats at a time, and provides physiologic characteristics C averaged over two or more heartbeats.

In one embodiment, Block 430 analyses one or more measurements to determine the quality of the measurement for diagnostic purposes. In one embodiment, Block 430 analyses one or more measurements to determine the quality of the measurement for diagnostic purposes. Thus, for example, in one embodiment, each ECG measurement is analyzed to determine if it is usable (a “good signal”) or not (a “bad signal”).

According to the invention, the noted determinations can be made based on predetermined values over a minimum and maximum heart period, a QRS minimum amplitude, and/or range of QRS complex widths. Further determinations can also be made on the OP measurement, according to a minimum heart period interval and minimum amplitude for systolic upstroke.

In an alternative embodiment, Block 430 includes one or more algorithms to remove beats with excessive drift in plethysmographic volume signal. Drift removal algorithms can include, without limitation, averaging signals from two or more successive beats and outlier detection filters, such as Hampel filters, that are adapted to remove beats where the overall stroke volume or intermediate variables, such as pulse transit time, show sufficient degree of inconsistency with temporally local beats.

According to the invention, Block 430 can be performed either digitally, for example, by a signal processor 72 or 94, e.g., using software filters, averagers and/or onset detectors, or by a combination of analogue and digital circuitry. In one embodiment, measurement(s) M comprise the output signals of signal processor 72 or 94. In an alternative embodiment, systems 200 and/or 300 include analogue circuits that are adapted to filter, rectify, or otherwise modify the output of ECG leads 90 and/or detector 60, 84 prior to converter 70, 88.

The analyzed information is then provided, as signal C, to Block 440, where one or more physiologic characteristics are displayed. In one embodiment, the physiologic characteristic(s) are visually displayed on a display, such as display 74 or 96.

In yet another embodiment, Block 430 stores information from previous heartbeats and compares measurements between heartbeats. This information can then be used for various diagnostic purposes. By way of example, heart rate variation and blood pressure variation (possibly cardiac output variation), can be used to better detect hypovolemia prior to anesthesia-patients react differently to anesthetic (i.e. crash). In addition, the valsalva maneuver can be used to assess autonomic reflex control of cardiovascular function by looking at the beat-to-beat fluctuations in heart rate, arterial pressure, and possibly cardiac output.

In one embodiment, the change in oxygen saturation is compared on a beat-to-beat basis as a function of changes in inspiratory oxygen percentage to derive a model based estimate of left ventricular ejection fraction. By way of example, a patient is given a breath of oxygen-enriched air. The resulting change in arterial oxygen saturation of blood (“SpO₂”) is analyzed over time as the enriched blood is mixed inside the heart and pumped out into the central and peripheral vasculature. The resulting rate of decay of the initially enriched SpO₂ is transformed to clinically useful “ejection fraction” in % by calibration against state of the art diagnostic technology, such as echocardiography.

In one embodiment, Block 430 includes a neural network that is trained by a set of data including measurement(s) M, demographic information D, and independently measured physiologic characteristics C. As is known in the art, neural networks are learning algorithms, wherein the algorithm is trained by using a series of inputs (i.e. pulse transit time and arterial volume) and output (i.e. cardiac output measured by Thermodilution) for a multitude of patients.

According to the invention, the neural network employs the noted training data to create a transform algorithm that relates the input to the output. The transform algorithm is then used on a separate set(s) of input and output data to validate system performance.

Thus, for example, in one embodiment, a training set of data is assembled that includes one or more cardiodynamic functions. The cardiodynamic functions are measured directly with accurate available devices to yield measurement(s) M, and demographic information D from a plurality of patients.

Block 430 is, in effect, an empirical or phenomenological model having many parameters. The measurement(s) M and demographic information D of the training set are provided to Block 430, and the model parameters are adjusted to minimize the error between the output of Block 430 and independently measured physiologic characteristics for the plurality of patients. A system so taught can then be used to provide an estimate of the physiologic characteristics C for other patients.

In another embodiment, Block 430 includes an analysis of measurement(s) M and demographic information D, according to a phenomenological model. The following discussion includes background for phenomenological models that estimate a pressure within or adjacent to the heart (or blood volume-time relationship proximate the heart) from measurements other than pressure measurements (including, but not limited to, ECG and/or OP measurements), and then compute cardiodynamic function based on the estimated pressures.

According to the invention, the method steps shown in FIG. 5 are preferably carried out as a transformation of input signals to cardiodynamic functions with or without the calculation of one or more variables that are, within the context of the algorithm, related to physical variables, such as estimates of blood pressures. Thus, for example, in one embodiment, intermediate variables are calculated that are, within the context of the algorithm, related to blood pressure, heart rates, pulse transit times, or some other physically identifiable quantity. In another embodiment, there may be no physically identifiable variable.

Thus, while one or more of the following embodiments may be described with respect to certain physically identifiable quantities, the present invention is not limited to any such representation of intermediate algorithm variables.

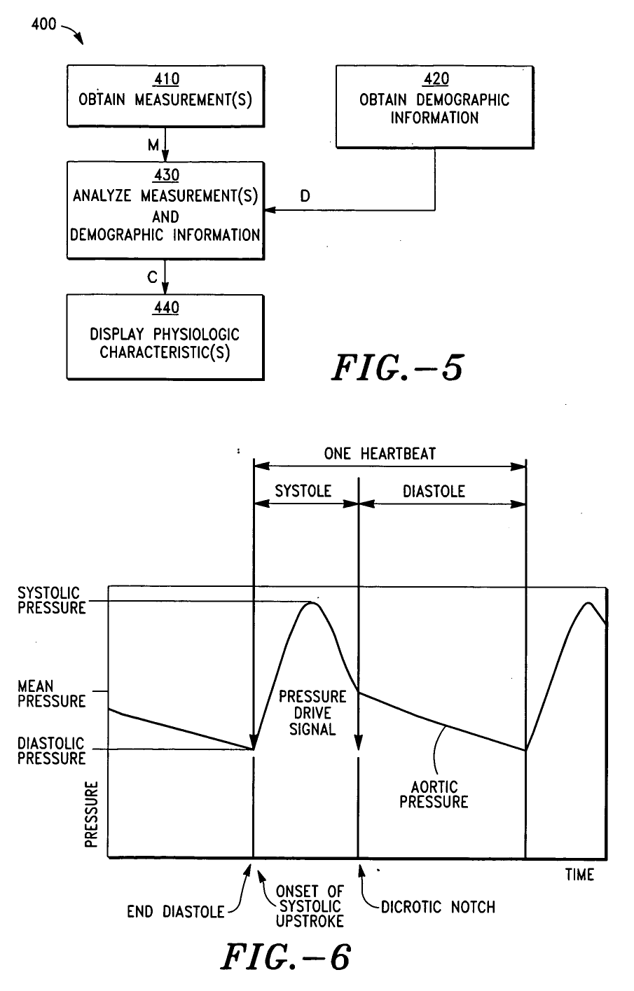

According to the invention, cardiodynamic function can be related to the variation of pressure within or near the heart. Referring now to FIG. 6, by way of illustration, there is shown a schematic of a pressure trace in a peripheral artery for more than one heartbeat. As is well known in the art, a heartbeat is one of a sequence of contractions (systole), resulting in an increase in pressure and expelling of blood into the arteries, and relaxations (diastole), resulting in a decrease in pressure and the filling of the heart chambers from the veins. The aortic pressure varies from a minimum of the diastolic pressure at or near the end of diastole to a maximum of the systolic pressure, which occurs during systole.

As shown in FIG. 6, the end of diastole and the beginning of systole occurs at or near the minimum pressure, while the beginning of diastole and end of systole occurs at the change of slope of the pressure curve, which is known as the dicrotic notch. Flow into the aorta occurs in the interval between the end of diastole and the dicrotic notch.

The work performed by the heart can be estimated as the integral of the difference between the arterial pressure and the diastolic pressure during the time between the end of diastole and dicrotic notch. This integral is the area designated “pressure drive signal”.

In one model for the flow of blood through the circulatory system the flow into the aorta from the left ventricle is first separated from flow out of the aorta into the periphery. The noted model and models similar thereto are set forth in Wesseling, et al., Adv. Cardiovas. Phys., vol. 5 (II), pp. 16-52 (1983); Wesseling, et al., J. Appl. Physiol., vol. 74, pp. 2566-2573 (1993); and Jansen, et al., Eur. Heart J., vol. 1 (Suppl), pp. 26-32 (1990); which are incorporated by reference herein. Using the noted models, measures of aortic blood pressure can be used to compute cardiodynamic function.

In several embodiments of the invention, aortic blood pressure is not measured directly. What is measured is the effect of the aortic pressure, which, according to the invention, comprises an optical photoplethysmograph (“OP”) signal that indicates the volume of blood flow through tissue that is distant from the heart. Specifically, as the heart contracts, pressure builds in the aorta, forcing blood through the arteries.

As is well known in the art, the flow of blood propagates through the circulatory system away from the heart. Thus, the maximum rate of volumetric blood flow in a finger lags, i.e. is delayed in time, from the maximum aortic volume. An OP signal indicating increased blood flow in the finger would accordingly occur some time after the heart begins forcing blood through the arteries.

The delay between the beginning systole in the heart and the increase in the OP measurement is referred to herein as the “pulse transit time” (“PTT”). Referring now to FIG. 7, by way of illustration, there is shown a schematic of an OP signal and an ECG signal corresponding to the aortic pressure trace of FIG. 6. FIG. 7 also shows a measure of the PTT.

In one embodiment, the PTT is determined from ECG and OP signals. In this embodiment, the functioning of the heart is monitored by the ECG. According to the invention, the onset of the QRS complex of the ECG signal, which is an indication of ventricular systole, is taken as one indicator of the beginning of an increase in blood pressure. The onset of systolic rise of the OP signal is the point where blood flow is increasing.

In one embodiment, the onset of systolic rise is taken at the time point when the OP signal is increasing at a defined rate, such as a fraction of the maximum rate of increase during that heartbeat. The PTT is then taken as the time delay between the onset of the QRS complex of the ECG signal and the onset of systolic uptake from the OP signal.

Alternatively, the PTT can be determined from demographic information—that is, as a function of age, gender, BSA, or other indicators using predetermined correlations from the general population.

One phenomenological model for blood flow that can be employed within the scope of the invention will now be presented. The following model is only one of several models that can be employed and, hence, is not meant to limit the scope of the invention.

In this model, the blood pressure is related to the stroke volume using pulse contour methods, which are based on the fluid mechanic analog of the electronic circuit equation:

p(t)=f(t)*z(t) (1)

where:
p(t)=pressure;
f(t)=volumetric flow; and
z(t)=impedance to flow at time t.

Equation 1 is applied to flow at the aorta as follows:

pₐ(t)−pᵥ(t)=f(t)*z(t) (2)

where:
pₐ(t)=arterial pressure;
pᵥ(t)=central venous pressure; and
z(t)=arterial impedance at time t.

Solving Equation 2 for flow:

$$f ( t ) = \frac{{p}_{a} ( t ) - {p}_{v} ( t )}{z ( t )} \qquad ( 3 )$$

According to the invention, Equation 3 can be used to calculate various cardiodynamic functions. For example, stroke volume (“SV”) is calculated for each beat by the following equation:

$$SV = {\int }_{{t}_{EndDiastole}}^{{t}_{DicroticNotch}} f ( t ) d t = {\int }_{{t}_{EndDiastole}}^{{t}_{DicroticNotch}} \frac{{p}_{a} ( t ) - {p}_{v} ( t )}{z ( t )} d t \qquad ( 4 )$$

where:
t_{EndDiastole}=the time at the end of diastole; and
t_{DicroticNotch}=the time at the dicrotic notch.

Alternatively, pᵥ(t) is approximated as the arterial pressure at the end of diastole, i.e. pᵥ(t)=p_{EndDiastole}.

In several embodiments of the invention, the pressures pₐ(t), pᵥ(t) and impedance z(t) are not measured, and the use of Equations 1-4 requires that a relationship be established between the variables in the equations and bodily measurements. Consider, for example, OP and ECG measurements. OP is a measure of the amount of blood within the tissue and the ECG is a measure of the electric activity of the heart. Blood pressures are generated in the heart as the result of electrical activity, and the pressures, such as systolic pressure, diminish in intensity and occur at increased delay times at increasing distances through the circulatory system.

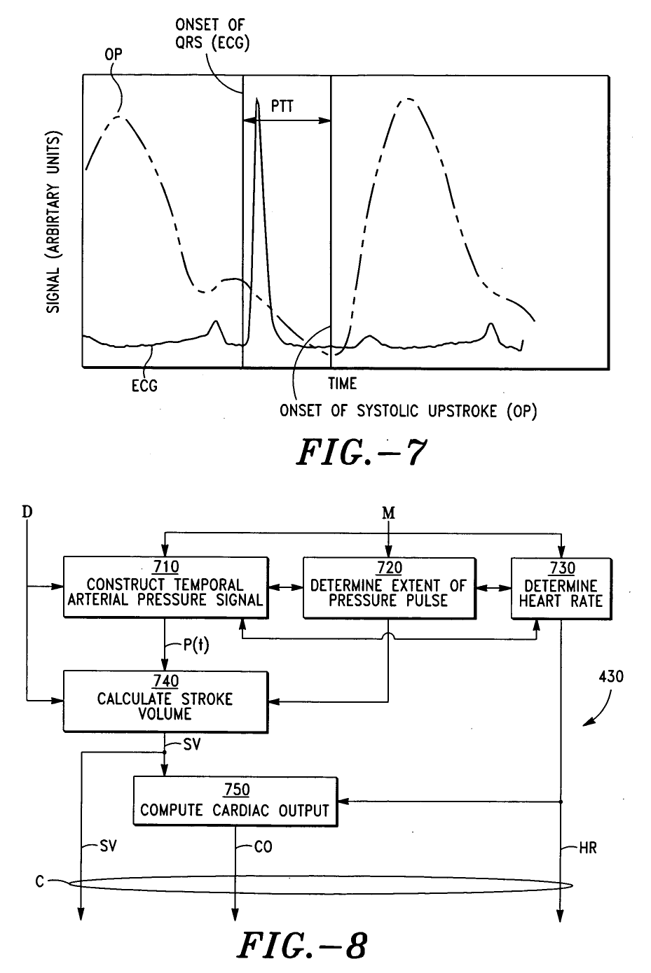

Referring now to FIG. 8, there is shown a flow diagram illustrating one embodiment of Block 430 for an arterial pressure-based phenomenological model, which can be generally similar to the embodiment illustrated in FIG. 5, except as further detailed below. More specifically, the flow diagram reflects one embodiment for relating measurements, including, but not limited to, those obtained as ECG and/or OP signals, to the pressure within the heart, and analyzing the related pressure to determine cardiodynamic functions.

As illustrated in FIG. 8, block 430 includes the following steps: Construct Temporal Arterial Pressure Signal (Block 710); Determine the Extent of Pressure Pulse (Block 720); Determine Heart Rate (Block 730); Calculate Stroke Volume (Block 740); and Compute Cardiac Output (Block 750).

In one embodiment of the invention, measurement(s) M includes at least one temporal measurement of blood flow through tissue, such as an OP signal from a pulse oximeter, and optionally includes one or more measurements of the function of the heart, such as an ECG measurement. Measurement M is provided as an input to Block 730, i.e. “Determine Heart Rate”, where the heart rate is determined. Measurement M, along with Demographics D, are provided as inputs to Block 710, i.e. “Construct Temporal Arterial Pressure Signal”, where the temporal measurements are converted to an estimated arterial pressure signal P(t).

Measurement M is also provided as an input to Block 720, “Determine Extent of Pressure Pulse”, where portions of pressure signal P(t) that correspond to systole are determined. With reference to Equation 4, the extent of pressure pulse includes the time of the end of diastole and the time of the dicrotic notch.

The output of Blocks 710 and 720, along with Demographics D, are provided as input to Block 740, i.e. “Calculate Stroke Volume”, where the data is analyzed, for example, according to Equation 4, to calculate stroke volume (“SV”). The stroke volume from Block 740 and the heart rate from Block 730 are provided as inputs to Block 750, i.e. “Compute Cardiac Output”, to compute the cardiac output (“CO”).

According to the invention one or more of the noted cardiodynamic functions, i.e. SV, heart rate and CO, are then provided as physiologic characteristics C.

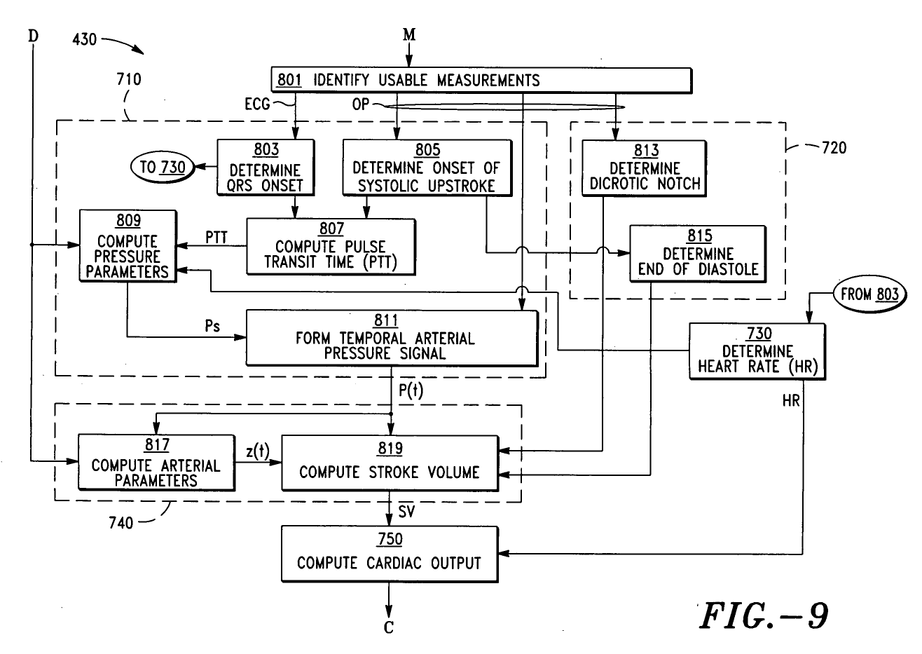

Referring now to FIG. 9, there is shown a flow diagram illustrating another embodiment of Block 430, which can be generally similar to the embodiments discussed above, except as further detailed below.

As illustrated in FIG. 9, Block 430 includes Block 801, i.e. “Identify Usable Measurements”, and Blocks 710, 720, 730, 740 and 750, as described subsequently. In Block 801, one or more OP and ECG signals are obtained as Measurement(s) M, where, in one embodiment, the signals are preferably initially analyzed for signal amplitude and integrity for the selection of best of several signals.

In one embodiment, the pre-qualifying of individual pulse signals is achieved by averaging two or more pulses while the patient is in a steady state, such as five to fifteen of high signal-to-noise or more per the generally accepted square root function rule, to allow for frequent updating. The derived initial template for qualifying a next incoming pulse then becomes a running average as new pulses are qualified and added. This method allows for signal-to-noise improvement by averaging, as well as for pre-qualifying next pulses by providing acceptance criteria of pulse contour, slope, amplitude, and other pulse-specific criteria.

In the event that an ECG lead or OP device comes loose, Block 801 can provide notification back to display 74 or 96 that a signal has been lost, and cease further signal processing. Alternatively, a patent may have two or more OP sensors, and Block 801 can determine which signal has maximum pulsatile amplitude and provide that signal for further processing.

Block 801 can also include spike filtering and/or band pass filtering for filtering of the signals. According to the invention, the filters can be implemented in hardware, software, or some combination thereof.

Block 710, i.e. Construct Temporal Arterial Signal, includes the following blocks: Determine QRS Onset (Block 803); Determine Onset of Systolic Upstroke (Block 805); Compute PTT (Block 807); Compute Pressure Parameters (Block 809); and Form Temporal Arterial Pressure Signal (Block 811).

In one embodiment of the invention, the input to Block 803 comprises an ECG signal, and the output is an indication of the time of the onset of the QRS complex. In one embodiment, the output of amplifier 92, which has been digitized by converter 88, is band pass filtered and then subjected to an amplitude detection algorithm.

In one embodiment, the ECG signal is sampled at 2000 Hz and stored in a circular buffer having a 5 second capacity. The stored data is analyzed to determine QRS onset and QRS peak. The stored date is first passed through a spike rejection filter, followed by a 5-40 Hz band pass filter, and then an amplitude detection algorithm is used to identify the QRS complex. The amplitude detection algorithm preferably comprises an adaptive bi-directional threshold detector, where the detection of matching bi-directional peaks above the threshold is indicative of a QRS complex.

In one embodiment, the time indicator comprises a time index that substantially corresponds to the moment of the identified QRS complex onset, i_{Q}.

In one embodiment, the input to Block 805 comprises one OP signal, and the output is an indication of the time of the onset of systolic upstroke. In one embodiment, the output of demodulator 68 or 82 that has been digitized by converter 70 or 88, respectively, is passed through a 5-60 Hz band pass filter followed by a nonlinear decaying threshold filter with debounce to detect the onset of systolic upstroke.

In one embodiment, the time indicator is a time index that substantially corresponds to the moment of the identified onset of systolic upstroke. First, the OP signal during the beat is analyzed to find the maximum of the product of the velocity and acceleration of the signal V, i.e.

$$max ( \frac{d V [ i ]}{d t} \frac{{d}^{2} V [ i ]}{d {t}^{2}} )$$

Next, the OP signal is analyzed to determine time index between the minimum OP signal and the systolic peak, where the product of the signal velocity and acceleration is 5% of the maximum value:

$$\frac{d V [ i ]}{d t} \frac{{d}^{2} V [ i ]}{d {t}^{2}} \leq 0.05 max ( \frac{d V [ i ]}{d t} \frac{{d}^{2} V [ i ]}{d {t}^{2}} )$$

In one embodiment, the time indicator comprises a time index that corresponds to a minimum index that meeting the criteria, i_{S}.

The input to Block 807 comprises the time indicators i_{Q} and i_{S} from Blocks 803 and 805, respectively, and the output comprises the PTT. Specifically, the PTT is computed as:

(i_{S}−i_{Q})Δt

where:
Δt=the time between digitized samples of the ECG and OP signals.

In one embodiment, the input to Block 809 comprises the PTT, heart rate, and Demographics D, and the output comprises a set of pressure parameters that are used in Block 811 to relate the OP signal to pressure P(t). In one embodiment, the pressure parameters comprise the minimum arterial pressure (P_{diastole}), the mean arterial pressure (Pₘₑₐₙ), and the maximum arterial pressure (P_{systole}).

In the following embodiment, Pₘₑₐₙ is estimated from PTT, heart rate, and Demographics D, and then P_{diastole} and P_{systole} are estimated using Pₘₑₐₙ and correlations of the pressure pulse. First, the mean arterial pressure is computed from the PTT, heart rate, and Demographics D of the person, including, but not limited to, combinations of the person's age, BSA, weight, and height.

In one embodiment, the mean arterial pressure for a patient is estimated from demographic correlations, independent measurements (or physiologic characteristics), such as PTT and heart rate, and modeling of blood flow through the heart and compliant arteries. It should, however, be noted that while it is possible to estimate a mean arterial pressure (“MAP”) from such data, it is also effective to estimate the MAP for the patient population without use of the PTT. In particular, it is not required to expressly calculate MPA to derive the cardiac output number from the collected patient data.

By employing PTT as an example of this approach, which is not meant to limit the scope of the invention, the transit of pressure from the heart to the location of an OP sensor will be considered. One model is based on a consideration of arterial wall compliance, which can be defined as:

Cp=Δv/Δp

where:
Cₚ=compliance at transmural pressure of p;
Δv=change in volume; and
Δp=the change in pressure.

For a constant length artery segment, Δv is proportional to Δa, i.e. the change in cross sectional area. Under conditions where no external pressure is applied to the artery Cₚ₌Cₐ; where Cₐ=arterial compliance.

The arterial wall has the greatest compliance at a transmural pressure of 0 mmHg. In essence, the artery at zero transmural pressure becomes vascularly unloaded or floppy. As is known in the art, the recognition that the arterial wall is vascularly unloaded at zero transmural pressure is the fundamental enabling principle associated with arterial cuffs and arterial tonometry systems measure arterial pressure.

In one model, PTT is related to the cross-sectional area of the aorta, aₐ, and the compliance of the arterial wall, Cₐ, as set forth below. The pulse wave velocity, PWV, which is the speed at which the pressure generated in the heart travels through the arteries, is determined by:

$$PWV = \sqrt{\frac{k * {a}_{a}}{ρ * {C}_{a}}} \\ where \text{:} \\ {a}_{a} = the cross \text{-} sectional area of the aorta ; \\ {C}_{a} = the compliance of the arterial wall ; \\ k = \frac{1333 mm Hg}{g / ( cm * {sec}^{2} )} , i . e . a unit conversion ; \\ and \\ ρ = 1.055 \frac{g}{ml} , i . e . the density of blood . \qquad ( 5 )$$

The average PWV is related to PTT as follows:

$$\underline{PWV} = \frac{Length}{( PTT - PEP )} \qquad ( 6 )$$

where:
Pre-Ejection Period (PEP)=the time between the onset of the QRS sequence and of the aortic valve; and
Length=the distance between aortic valve and the digit.

PEP is calculated from average heart rate patient demographics, i.e.

PEP(female)=0.133−(HR*(0.0004)) (7-F)

PEP(male)=0.131−(HR*(0.0004)) (7-M)

Length(male)=0.426*Height(cm)*(1.3) (8-M)

Length(female)=0.412*Height(cm)*(1.3) (8-F)

where factor 1.3 provides a conversion from arm length to (arm length+chest length).

Since PWV is a function of cross-sectional area, PWV is not constant throughout the arterial tree. Thus, to approximate aortic pulse wave velocity, PWVₐ, PWV must be scaled appropriately:

$${PWV}_{a} = \frac{\underline{553 + 5.10 * Age}}{817 + 0.61 * Age} * ( \underline{PWV} ) \qquad ( 9 )$$

Equations 5 and 9 provide a relationship between the PTT, aₐ, and Cₐ and demographic variables as follows:

$$\frac{\underline{553 + 5.10 * Age}}{817 + 0.61 * Age} * \frac{Length}{( PTT - PEP )} = \sqrt{\frac{k * {a}_{a}}{ρ * {C}_{a}}} . \qquad ( 10 )$$

where:
Length and PEP are obtained from Equations 7 and 8.

The arterial cross section is a complicated function of pressure. Thus, further analysis is required to determine the mean arterial pressure.

One approach is to consider the relationship between patient demographics and the pressure-volume curves in excised tissue, including the aorta. This approach yields a series of interrelated equations as follows. First, the following intermediate parameters are calculated:

P₀(female)=72−(0.89*Age)mmHg (1-F)

P₀(male)=76−(0.89*Age)mmHg (1-M)

P₁=57.0−(0.44*Age)mmHg (12)

where:
P₀=an intermediate pressure offset term used to calculate pressure-cross sectional area curve;
Age=patient's age in years; and
P₁=an intermediate pressure scaling term used to calculate pressure-cross sectional area curve.

Equations 11 and 12 are combined to form:

$${P}_{term} = \frac{{p}_{a} - {p}_{0}}{{p}_{1}} \qquad ( 13 )$$

where:
Pₐ=the mean arterial pressure; and
Pₜₑᵣₘ=the intermediate pressure term.

According to the invention, the maximum cross-sectional area of the aorta, Aₘₐₓ, is correlated as:

$$\begin{matrix}{A}_{max} ( female ) = \frac{\begin{matrix}0.98 * ( 1 - ( 60 - Age ) * 0.003 ) * \\ ( ( 2.4 * {BSA}_{M} ) + 0.5 + π * {( 2.4 * {BSA}_{M} + 1.06 )}^{2} )\end{matrix}}{2} \\ ( designated hereinafter as Eq . 14 \text{-} F ) & \\ {A}_{max} ( male ) = \frac{\begin{matrix}1.02 * ( 1 - ( 60 - Age ) * 0.003 ) * \\ ( ( 2.4 * {BSA}_{M} ) + 0.5 + π * {( 2.4 * {BSA}_{M} + 1.06 )}^{2} )\end{matrix}}{2} \\ ( designated hereinafter as Eq . 14 \text{-} M ) & \end{matrix}$$

where:
BSA=the patient's body surface area.

The BSA can either be measured or provided though a demographic correlation based on weight and height, as shown in FIG. 10.

In one embodiment of the invention, BSA_{M} includes a further adjustment, intended to retard the increases in Aₘₐₓ in obese patients. According to one embodiment of the invention, the BSA_{M} adjustment comprises the following:

The “ideal” weight, W₁, for an individual of a given height is initially calculated as follows:

$${W}_{1} = \frac{\begin{matrix}( ( 50 + 2.3 ( Ht . - 60 inches * 2.54 cm / inch ) ) + \\ ( Ht . * 0.534 - 17.36 ) )\end{matrix}}{2}$$

where:
Ht.=Height (cm).

The BSA is then adjusted as follows:
a) For BMI<=25;

BSAₓ=BSA
b) For BMI>25 (which produces a slight retardation in the increase for severely obese individuals);

BSAₓ=(Max((10−BMI/5),1)*BSA+BSA_{ideal})/(1+(Max((10−BMI/5),1))

where:
BSA_{ideal}=BSA calculated for height and W₁.

The maximum aortic area, A_{MAX}, can then be calculated as follows:

Aₘₐₓ=(((2.4*BSAₓ)+0.5+pi((0.845*BSAₓ)+1.06)²)/2)*(1−((60−Age)*0.003))*1.02

According to one embodiment of the invention, Equations 11-14 are used to calculate the aortic cross-sectional area, aₐ, and arterial compliance, Cₐ, as follows:

$$\begin{matrix}{a}_{a} = {A}_{max} * ( 0.5 + {tan}^{- 1} ( {P}_{term} ) ) & ( 15 ) \\ {C}_{a} = \frac{{A}_{max}}{( ( 1 + {P}_{term}^{2} ) * {π}^{2} )} & ( 16 )\end{matrix}$$

Using Equations 13, 15 and 16, a relationship between arterial pressure, pₐ, aortic cross sectional area and arterial compliance is obtained.

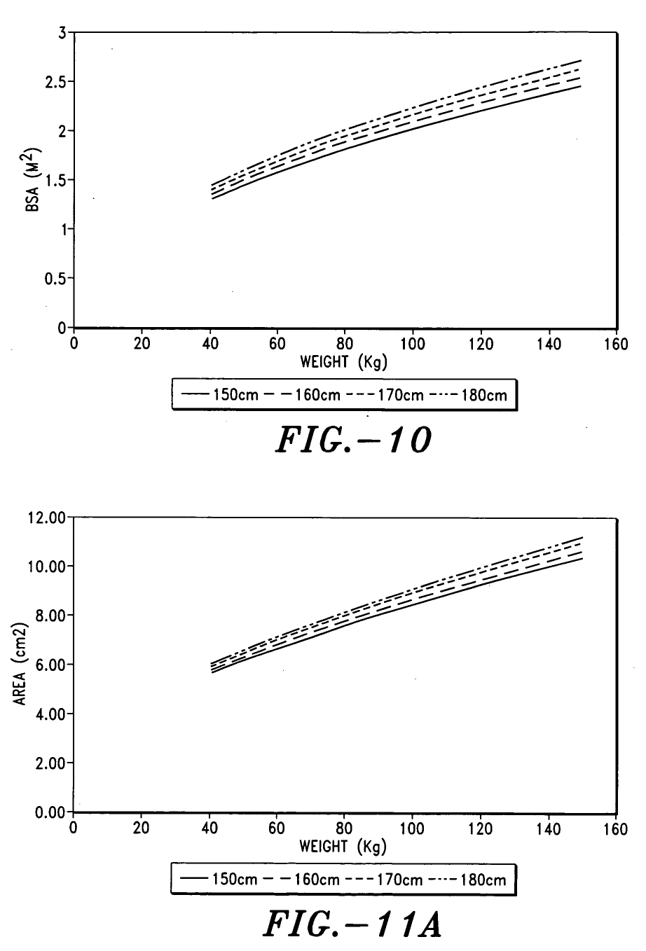

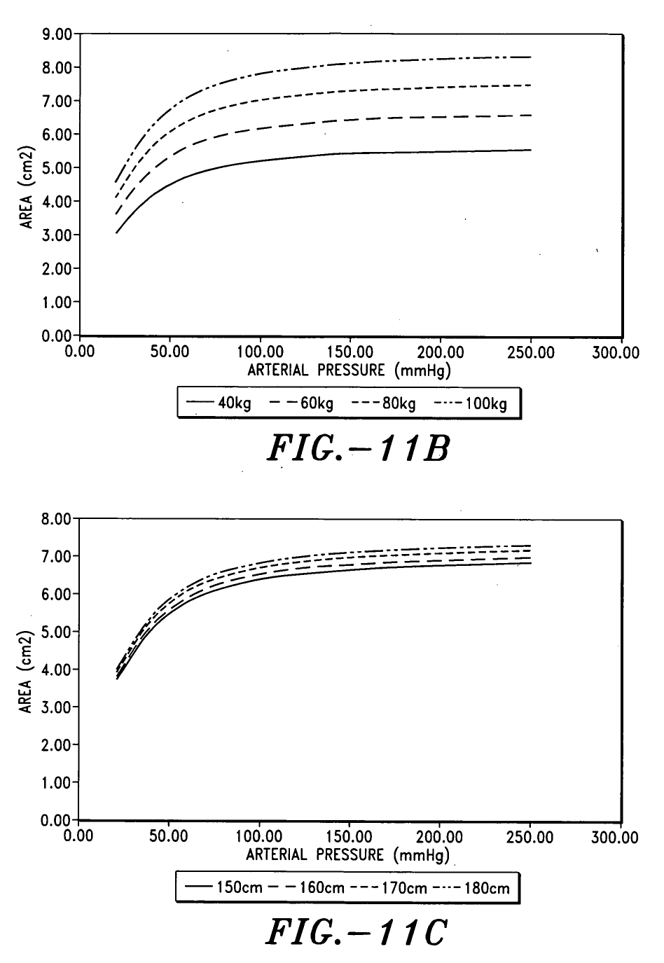

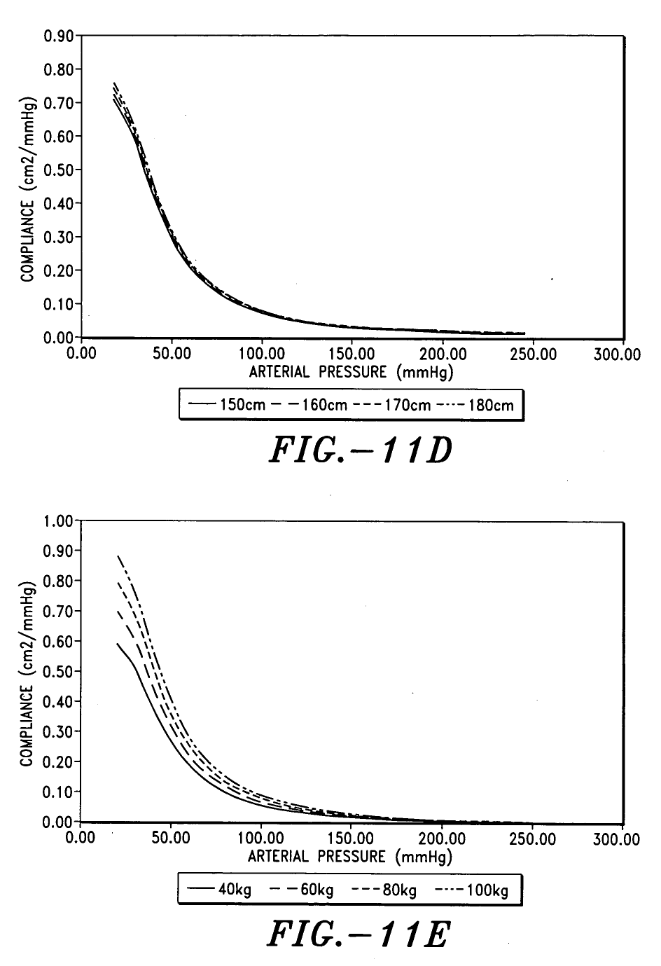

Representative graphical illustrations demonstrating this relationship is shown in FIGS. 11A-11E for a female of a specific age (in this instance, 65 years); where FIG. 11A illustrates Aₘₐₓ as a function of weight, with a family of curves for different heights; FIG. 11B illustrates Aₘₐₓ as a function of pₐ, with a family of curves for different heights; FIG. 11C illustrates Aₘₐₓ as a function of pₐ of a specific height, with a family of curves for different weights; FIG. 11D illustrates Cₐ as a function of pₐ for a specific height, with a family of curves for different weights; and FIG. 11E is Aₘₐₓ as a function of pₐ for of a specific height, with a family of curves for different weights.

According to the invention, Equations 13, 15 and 16, or graphical representations, such as those shown in FIGS. 10A-10E can be used to determine Cₐ, aₐ, and pₐ using demographic information, such as the age, gender, weight, height, and BSA.

Combining Equations 10, 15, and 16, along with demographic information and the heart rate in the demographic correlations of Equations 7, 8, 11, 12, 14, provides a solution for three unknown parameters: Cₐ, aₐ and pₐ; where pₐ is assumed to equate to mean arterial pressure.

Although difficult to solve in a closed form solution, the interactions between the noted parameters plus PTT can be readily solved through a series of x-y transforms or look up tables.

Next, the maximum arterial pressure, P_{systole}, and minimum arterial pressure, P_{systole}, are estimated. It is assumed that there is a general relationship between the minimum, maximum and mean arterial pressures. On such relationship is the “⅓-⅔” rule, which postulates that:

$${P}_{mean} = \frac{1}{3} {P}_{systole} + \frac{2}{3} {P}_{diastole} \qquad ( 17 )$$

In addition, it is assumed that the pressure pulse, P_{systole}−P_{diastole}, is one half of the difference between the mean arterial pressure and the central venous pressure, P_{CV}, which is assumed to be 12 mmHg. This provides:

P_{systole}−P_{diastole}=a(Pₘₑₐₙ−P_{CV}) (18)

where:
a=0.5; and
P_{CV}=12 mmHg.

Equations 17 and 18 can be solved to provide the maximum arterial pressure, P_{systole}, and the minimum arterial pressure, P_{diastole}.

Referring back to FIG. 9, the input to Block 811 comprises the pressure parameters from Block 809 and an OP signal, V(t), illustrated, for example, by the OP signal shown in FIG. 7. The OP signal, V(t), is a measure of blood flow in the vicinity of the OP sensor (for example, emitters 76 and 78 and detector 84 of FIG. 2, or emitters 56 and 58 and detector 60), which varies from a minimum value, Vₘᵢₙ, to a maximum value, Vₘₐₓ, and has a mean value, Vₘₑₐₙ, as averaged over a heartbeat.

In one embodiment, Block 811 relates P_{diastole}, Pₘₑₐₙ, and P_{systole} to V(t) as follows. First, an OP signal, V(t), is obtained over a period of time corresponding one heat beat. The time may correspond, for example, to the time period between consecutive onsets of systolic upstroke, e.g., from time t₁ to time t₂.

The average signal is preferably calculated from:

Vₘᵢₙ=∫ₜ₁^{t2}V(t)dt/(t2−t1) (19)

In one embodiment, the signals comprise digitized signals, and a digital signal processing equivalent of this equation is used. Further analysis of V(t) from t₁ to t₂ results in a value for the minimum value, Vₘᵢₙ, and the maximum value Vₘₐₓ during the beat.

Next, a multi-dimensional fit is performed for the estimated arterial pressure signal P(t), such that the minimum arterial pressure, P_{diastole}, corresponds to the minimum OP signal value, Vₘᵢₙ, the mean arterial pressure, Pₘₑₐₙ, corresponds to the mean OP signal value, Vₘₑₐₙ, and the maximum arterial pressure, P_{systole}, corresponds to the maximum OP signal value, Vₘₐₓ. The resulting fit is used to map each value of V(t) to a value of P(t).

In one embodiment, the fit comprises a second order polynomial. Thus, for example, a least squares fit of the Equation 20, below:

P(t)=P_{diastole}+a(V(t)−Vₘᵢₙ)+b(V(t)−Vₘᵢₙ)² (20)

Equation 20 can be employed for measurements over a heartbeat, to the minimum, mean, and maximum values of P and V, and provides fitting parameters a and b.

Equation 20 is then used to convert each OP signal value to an estimate of the arterial pressure. It is understood that this estimate is shifted in time from the actual arterial pressure by PTT. However, since the calculations are performed over a heartbeat, this delay does not affect the calculation.

In an alternative embodiment, PTT is derived from demographic correlations. In accordance with one embodiment of the invention, the average PTT of a patient population is preferably established in a calibration phase. The fixed average PTT is then used thereafter for any new patient that falls within the calibration population.

The noted embodiment eliminates Blocks 803 and 807, and thus permits Block 710 to provide P(t) without an ECG measurement.

As illustrated in FIG. 9, Block 720, i.e. Determine the Extent of Pressure Pulse, includes the following blocks: Determine Dicrotic Notch (Block 813); and Determine End of Diastole (Block 815). The input to Blocks 813 and 815 preferably comprises an OP signal. The output of Block 813 comprises the time or time index that corresponds to the dicrotic notch during the current beat. The output of Block 815 comprises the time or time index that corresponds to the end of diastole.

According to the invention, Block 813 includes an algorithm to determine the change in slope of V(t), where V(t) is the measured volume at time t, that corresponds to the dicrotic notch. The dicrotic notch occurs at a transition from a negative acceleration (d²V/dt²<0) to a positive acceleration (d²V/dt²<0) with the window of time corresponding to peak systole and minimum diastole. In one embodiment, the algorithm first identifies peak systole and minimum diastole, calculates d²V/dt² for each point within this window, and then identifies transitions from negative to positive acceleration.

In an alternative embodiment of the invention, a “change in slope” method is employed to determine the end of the arterial ejection period. The change in slope method operates on the data between peak systole and subsequent minimum diastole prior to subsequent systolic upstroke.

In one embodiment, the change in slope method identifies the time point k, where jointly:
a) the 2nd derivative of volume is maximum, i.e. max (d²V(k)/d²t);
b) the function of (V,k,n)=weighted score determined by the number of contiguous samples around k,
   where:
acceleration is non negative, and
n=the sample step; and
c) the function of ((V(k),Vₘᵢₙ,Vₘₐₓ)=weighted score using the logic that the volume at the end of the ejection period is preferably not near Vₘᵢₙ or Vₘₐₓ during the beat.

By combining the elements above, the end of arteriole ejection time stamp can be determined.

In general, the shape of an OP signal is less distinct than the shape of the aortic pressure curve, and becomes less so as one proceeds along the circulation system. To aid in the detection of the dicrotic notch, in one embodiment, a score based on four separate tests is used to determine the presence of the dicrotic notch.

For each transition, the test is performed on that data point following the transition having a local maximum acceleration, i.e.

(V[k]+V[k−0.1 seconds]−2×V[k−0.05 seconds])

where:
V[k] denotes the value of V at time (sample) index k;
V[k−0.05 seconds]=the value at 0.05 seconds before the time corresponding to index k; and
V[k−0.1 seconds]=the value at 0.1 seconds before the time corresponding to index k.

According to the invention, two scores are calculated for each tested transition point. These scores are based on a Duration Test and a Ratio Test.

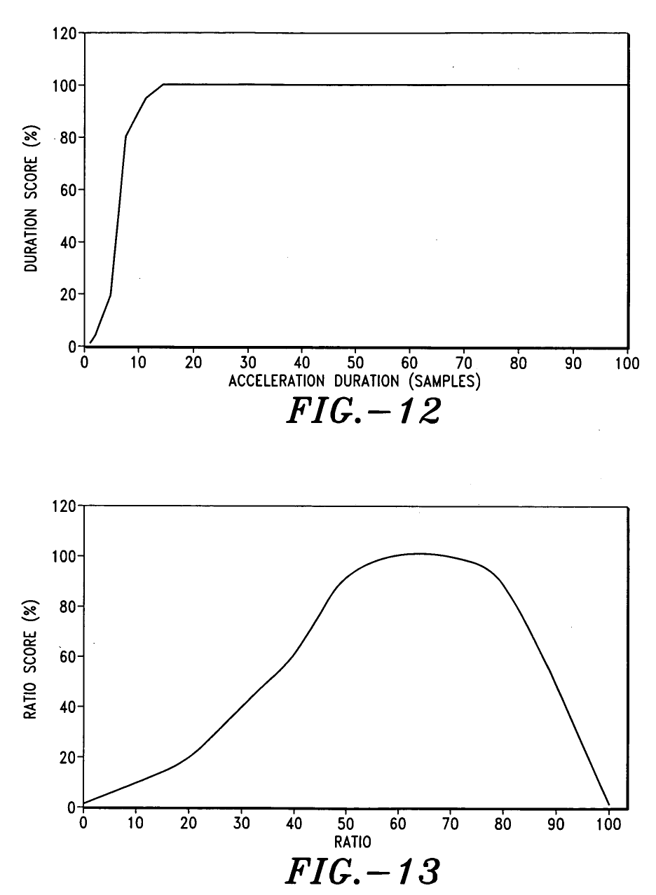

In the Duration Test the score comprises a sequential number of contiguous data samples, wherein the acceleration is positive. FIG. 12 illustrates the Duration SCORE (y-axis) as a function of number of contiguous samples (x-axis), wherein the acceleration is positive. This Duration SCORE substantially eliminates the possibility of short spikes in the volume waveshape being misinterpreted as the dicrotic notch.

In the Ratio Test the score comprises the ratio: (Test point Volume−Diastolic Volume) to (Peak Systolic Volume−Diastolic Volume). FIG. 13 illustrates the Ratio SCORE (y-axis) as a function of the ratio (x-axis). This score substantially eliminates the possibility of regions to near the systolic peak volume or too near end diastolic volume being misinterpreted as the dicrotic notch.

Since the maximum acceleration point is determined using a realitively large window (0.1 seconds), the final dicrotic point is refined by searching a short region (0.05 seconds) prior to previously determined max acceleration for the maximum acceleration over a much narrower region: Max, (P[k+0.01 seconds+P[k−0.01 seconds]−2×P[k]). It should be noted that this acceleration calculation is also centered around the refined dicrotic notch point.

In Block 815 the end of diastole is determined. In one embodiment, the end of diastole is assumed to occur at the time index just before the onset of systolic uptake, or i_{S}−1.

Block 730, i.e. Determine Heart Rate, receives input from Block 803, where each QRS complex is detected. The time between successive heartbeats is designated T_{HB}, and the heart rate is designated 1/T_{HB}.

In an alternative embodiment, the heart rate is assumed to be the same as the pulse rate, as determined by the OP. The determination of pulse rate from and OP or pulse oximeter probe is well known in the field.

In addition, if the PTT is also derived from demographic correlations, as discussed previously as an alternative embodiment, then no ECG measurement is needed for Block 430, and cardiodynamic functions can be estimated from only an OP signal.

Referring back to FIG. 9, Block 740, i.e. Calculate Stroke Volume, includes the following blocks: Compute Arterial Parameters (Block 817); and Compute Stroke Volume (Block 819). In one embodiment, Block 740 uses the estimated arterial pressure signal P(t) and demographics D to compute SV using Equation 4:

$$SV = {\int }_{{t}_{EndDiastole}}^{{t}_{DicroticNotch}} f ( t ) d t = {\int }_{{t}_{EndDiastole}}^{{t}_{DicroticNotch}} \frac{{p}_{a} ( t ) - {p}_{v} ( t ) }{z ( t )} d t \qquad ( 4 )$$

The arterial pressure, pₐ(t), comprises the estimated arterial pressure p(t) from Block 811. The pressure pᵥ(t) comprises the arterial pressure at the end of diastole, i.e. pᵥ(t)=p(t=End Diastole).

The impedance z(t) is provided by:

$$z ( t ) = \sqrt{\frac{ρ}{{a}_{a} ( t ) {C}_{a} ( t )}} \qquad ( 20 )$$

where:
aₐ is determined by Equation 15; and
Cₐ is determined by Equation 16.

In Block 817, Equations 15 and 16 are first solved for the estimated arterial pressure. Thus, for example, pressure, pₐ, in Equation 13 is taken to be the current estimated pressure, p(t). Demographics are used in Equation 14 and these values are used in Equations 15 and 16 to compute aₐ and Cₐ. Next, Equation 20 is used to calculate z(t).

In Block 819, z(t) from Block 817 and p(t) from Block 811, along with the bound of integration from Blocks 813 (i.e. the time of occurrence of the dicrotic notch) and 815 (the time of occurrence of the end of diastole) are used in Equation 4 to compute SV. It is understood that the measurements being used are sampled, digitized waveforms, and thus a digital equivalent of the integral is used.

In Block 750 the cardiac output is computed. Block 750 accepts the output of Block 819, i.e. SV, and the output of Block 730, i.e. heart rate, and computes the cardiac output as follows:

$$CO = \frac{SV ( liters ) * 60 ( sec ) / 1 ( min )}{HeartPeriod ( sec )} \qquad ( 21 )$$

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

What is claimed is:

1. A method of determining a physiologic characteristic associated with cardiac function in a subject, comprising the steps of:
providing at least one non-invasive measurement representing a physiological parameter of the subject, said non-invasive measurement comprising electromagnetic radiation absorption;
determining at least one temporal plethysmographic value from said electromagnetic radiation absorption;
providing demographic information, said demographic information including information reflecting the subject's physical condition; and
determining at least one physiologic characteristic from said temporal plethysmographic value and demographic information by using a predetermined phenomenological model, said model being adapted to provide an estimate of a blood volume-time relationship proximate the heart from non-pressure related measurements and compute at least one physiologic characteristic associated with cardiac function based on said estimated blood volume-time relationship.

2. The method of claim 1, wherein said subject's physical condition information is selected from the group consisting of the subject's weight, height, age, gender and body mass index.

3. The method of claim 1, wherein said subject's physical condition information is selected from the group consisting of a disease condition, disease diagnosis and therapy for a disease.

4. The method of claim 1, wherein said physiologic characteristic is selected from the group consisting of heart rate, pulse transit time, wave velocity, central blood flow, peripheral blood flow, perfusion, contractility and vasoconstriction.

5. The method of claim 1, wherein said cardiac function is selected from the group consisting of stroke volume, cardiac output and cardiac index.

6. The method of claim 4, wherein said blood flow is determined by calculating for blood pressure (p(t)) according to the formula
p(t)=f(t)*z(t),
wherein p(t) represents pressure, f(t) represents volumetric flow, and z(t) represents impedance to flow at time t, and calculating for arterial volumetric flow at the aorta according to the formula
pₐ(t)−pᵥ(t)=f(t)*z(t),
wherein pₐ(t) represents arterial pressure, pᵥ(t) represents central venous pressure, and z(t) represents arterial impedance at time t, and calculating for volumetric flow according to the formula
$$f ( t ) = \frac{{p}_{a} ( t ) - {p}_{v} ( t )}{z ( t )} .$$

7. The method of claim 5, wherein said stroke volume (SV) is calculated according to the formula
$$SV = {\int }_{{t}_{EndDiastole}}^{{t}_{DicroticNotch}} f ( t ) d t = {\int }_{{t}_{EndDiastole}}^{{t}_{DicroticNotch}} \frac{{p}_{a} ( t ) - {p}_{v} ( t ) }{z ( t )} d t ,$$
wherein, t_{EndDiastole} represents the time at the end of diastole and t_{DicroticNotch} represents the time at the dicrotic notch.

8. The method of claim 7, wherein said pᵥ(t) is approximated as the arterial pressure at the end of diastole.

9. The method of claim 1, wherein said electromagnetic radiation absorption is measured on at least one location on the subject's body selected from an arm, hand, finger, ear canal, ear lobe, foot, toe, nare, tongue, back, neck and forehead.

10. The method of claim 1, wherein said electromagnetic radiation absorption is measured on multiple locations on the subject's body selected from an arm, hand, finger, ear canal, ear lobe, foot, toe, nare, tongue, back, neck and forehead.

11. The method of claim 1, wherein said electromagnetic radiation absorption is measured on at least one location on the subject's body selected from the group consisting of the neck and forehead.

12. The method of claim 1, wherein said physiologic characteristic is visually displayed on a display medium.

13. A method of determining a physiologic characteristic associated with cardiac function in a subject, comprising the steps of:
providing at least first and second non-invasive measurements representing physiological parameters of the subject, said first non-invasive measurement comprising an electrical measurement of the heart, said second non-invasive measurement comprising electromagnetic radiation absorption;
determining at least one temporal relationship from said electrical measurement of the heart;
determining at least one temporal plethysmographic value from said electromagnetic radiation absorption;
providing demographic information, said demographic information including information reflecting the subject's physical condition; and
determining at least one physiologic characteristic from said temporal relationship, temporal plethysmographic value and demographic information by using a predetermined phenomenological model, said model being adapted to provide an estimate a blood volume-time relationship proximate the heart from non-pressure related measurements and compute at least one physiologic characteristic associated with cardiac function based on said estimated blood volume-time relationship.

14. The method of claim 13, wherein said subject's physical condition information is selected from the group consisting of the subject's weight, height, age, gender and body mass index.

15. The method of claim 13, wherein said subject's physical condition information is selected from the group consisting of a disease condition, disease diagnosis and therapy for a disease.

16. The method of claim 13, wherein said physiologic characteristic is selected from the group consisting of heart rate, pulse transit time, wave velocity, central blood flow, peripheral blood flow, perfusion, contractility and vasoconstriction.

17. The method of claim 13, wherein said cardiac function is selected from the group consisting of stroke volume, cardiac output and cardiac index.

18. The method of claim 16, wherein said central blood flow is determined by calculating for blood pressure (p(t)) according to the formula
p(t)=f(t)*z(t),
wherein p(t) represents pressure, f(t) represents volumetric flow, and z(t) represents impedance to flow at time t, and calculating for arterial volumetric flow at the aorta according to the formula
pₐ(t)−pᵥ(t)=f(t)*z(t),
wherein pₐ(t) represents arterial pressure, pᵥ(t) represents central venous pressure, and z(t) represents arterial impedance at time t, and calculating for volumetric flow according to the formula
$$f ( t ) = \frac{{p}_{a} ( t ) - {p}_{v} ( t )}{z ( t )} .$$

19. The method of claim 17, wherein said stroke volume (SV) is calculated according to the formula
$$SV = {\int }_{{t}_{EndDiastole}}^{{t}_{DicroticNotch}} f ( t ) d t = {\int }_{{t}_{EndDiastole}}^{{t}_{DicroticNotch}} \frac{{p}_{a} ( t ) - {p}_{v} ( t ) }{z ( t )} d t ,$$
wherein, t_{EndDiastole} represents the time at the end of diastole and t_{DicroticNotch} represents the time at the dicrotic notch.

20. The method of claim 19, wherein said pᵥ(t) is approximated as the arterial pressure at the end of diastole.

21. The method of claim 13, wherein said electrical measurement of the heart comprises electrical potential of the heart.

22. The method of claim 21, wherein said electrical potential of the heart is measured by an electrocardiogram.

23. The method of claim 13, wherein said electromagnetic radiation absorption is measured on at least one location on the subject's body selected from an arm, hand, finger, ear canal, ear lobe, foot, toe, nare, tongue, back, neck and forehead.

24. The method of claim 13, wherein said electromagnetic radiation absorption is measured on multiple locations on the subject's body selected from an arm, hand, finger, ear canal, ear lobe, foot, toe, nare, tongue, back, neck and forehead.

25. The method of claim 13, wherein said electromagnetic radiation absorption is measured on at least one location on the subject's body selected from the group consisting of the neck and forehead.

26. The method of claim 13, wherein said first and second non-invasive measurements comprise time varying measurements.

27. The method of claim 13, wherein said physiologic characteristic is visually displayed on a display medium.

28. A method of determining a physiologic characteristic associated with cardiac function in a subject, comprising the steps of:
providing at least one non-invasive measurement representing a physiological parameter of the subject, said non-invasive measurement comprising electromagnetic radiation absorption;
determining at least one temporal plethysmographic value from said electromagnetic radiation absorption;
performing non-linear scaling of said temporal plethysmographic value according to a physiological parameter selected from the group consisting of minimum blood pressure, maximum blood pressure and a predetermined blood pressure, said predetermined blood pressure being greater than said minimum blood pressure and less then said maximum blood pressure; and
determining a time-varied blood pressure from said scaled temporal plethysmographic value.

29. The method of claim 28, wherein a plurality of electromagnetic radiation absorption measurements are provided.

30. The method of claim 29, wherein a plurality of temporal plethysmographic values is determined from said plurality of electromagnetic radiation absorption measurements.

31. The method of claim 19, wherein said plurality of temporal plethysmographic values is scaled according to at least one of said physiological parameters.

32. A method of determining a physiologic characteristic associated with cardiac function in a subject, comprising the steps of:
providing at least first and second non-invasive measurements representing physiological parameters of the subject, said first non-invasive measurement comprising an electrical measurement of the heart, said second non-invasive measurement comprising electromagnetic radiation absorption;
providing demographic information, said demographic information including information reflecting the subject's physical condition;
determining temporal arterial pressure from said first and second non-invasive measurements and said demographic information;
determining the extent of a pressure pulse from said first and second non-invasive measurements;
determining heart rate from said first and second non-invasive measurements;
determining stroke volume from said temporal arterial pressure, extent of a pressure pulse and demographic information; and
determining cardiac output from said stroke volume and said heart rate.

33. The method of claim 32, wherein said subject's physical condition information is selected from the group consisting of the subject's weight, height, age, gender and body mass index.

34. The method of claim 32, wherein said subject's physical condition information is selected from the group consisting of a disease condition, disease diagnosis and therapy for a disease.

35. The method of claim 32, wherein a plurality of first and second non-invasive measurements are provided.

36. The method of claim 35, including the step of pre-qualifying said first and second non-invasive measurements.

37. The method of claim 36, wherein said pre-qualifying comprises averaging said plurality of said first non-invasive measurements to provide mean first non-invasive measurements.

38. The method of claim 36, wherein said pre-qualifying comprises selecting a third non-invasive measurement from said plurality of second non-invasive measurements, said third non-invasive measurement having a maximum pulsatile amplitude.

39. The method of claim 36, wherein said pre-qualifying comprises filtering of said plurality of first and second non-invasive measurements.

40. The method of claim 32, wherein said step of determining temporal arterial pressure includes the steps of determining QRS onset, determining the onset of systolic upstroke, determining pulse transit time and determining selective blood pressure parameters.

41. The method of claim 40, wherein said step of determining QRS onset provides the time of the onset of the QRS complex.

42. The method of claim 41, wherein said QRS onset time indicator comprises a QRS time index that substantially corresponds to the moment of said onset of the QRS complex.

43. The method of claim 40, wherein said step of determining the onset of systolic upstroke provides the time of the onset of systolic upstroke.

44. The method of claim 43, wherein said systolic upstroke time indicator comprises a systolic upstroke time index that substantially corresponds to the moment of said onset of systolic upstroke.

45. The method of claim 44, wherein said pulse transit time is determined from said QRS and systolic time indices.

46. The method of claim 40, wherein said blood pressure parameters are selected from the group consisting of minimum arterial pressure (P_{diastole}), mean arterial pressure (Pₘₑₐₙ), and maximum arterial pressure (P_{systole}).

47. The method of claim 46, wherein said mean arterial pressure (Pₘₑₐₙ) is determined from said pulse transit time, heart rate and demographic information.

48. The method of claim 47, wherein said minimum arterial pressure (P_{diastole}) and maximum arterial pressure (P_{systole}) are determined from said mean arterial pressure (Pₘₑₐₙ) and correlations of said pressure pulse.

49. The method of claim 46, wherein said mean arterial pressure (Pₘₑₐₙ) is determined from at least one physiologic characteristic, said demographic information and a blood flow model adapted to provide a model of blood flow through the heart and compliant arteries, said physiologic characteristic being selected from the group consisting of heart rate, pulse transit time, wave velocity, central blood flow, peripheral blood flow, perfusion, contractility and vasoconstriction.

50. The method of claim 32, wherein determining the extent of a pressure pulse includes determining portions of an arterial pressure signal that corresponds to systole.

51. The method of claim 32, wherein said step of determining the extent of the pressure pulse includes the steps of determining the dicrotic notch and end of diastole.

52. The method of claim 51, wherein said step of determining the end of diastole provides a time index that corresponds to the end of diastole during said predetermined heart beat.

53. The method of claim 51, wherein said step of determining the dicrotic notch provides a time index that corresponds to the dicrotic notch during a predetermined heart beat.

54. The method of claim 53, wherein said step of determining said dicrotic notch includes the use of an algorithm to determine the change in slope of a signal representing measure of blood flow (V(t)), wherein V(t) comprises the measured volume at time (t) that corresponds to said dicrotic notch.

55. The method of claim 54, wherein said dicrotic notch occurs at a transition from negative acceleration (d²V/dt²<0) to positive acceleration (d²V/dt²<0), said transition having a window of time corresponding to peak systole and minimum diastole.

56. The method of claim 55, wherein said algorithm identifies said peak systole and minimum diastole and calculates d²V/dt² for each point within said time window.

57. The method of claim 55, wherein said algorithm further identifies transitions from said negative to positive acceleration.

58. The method of claim 32, wherein said stroke volume (SV) is calculated according to the formula
$$SV = {\int }_{{t}_{EndDiastole}}^{{t}_{DicroticNotch}} f ( t ) d t = {\int }_{{t}_{EndDiastole}}^{{t}_{DicroticNotch}} \frac{{p}_{a} ( t ) - {p}_{v} ( t ) }{z ( t )} d t ,$$
wherein SV represents stroke volume, pₐ(t) represents arterial pressure, pᵥ(t) represents arterial pressure at the end of diastole, t_{EndDiastole} represents the time at the end of diastole and t_{DicroticNotch} represents the time at the dicrotic notch, and wherein z(t) is calculated from a formula
$$z ( t ) = \sqrt{\frac{ρ}{{a}_{a} ( t ) {C}_{a} ( t )}} ,$$
wherein the aortic cross-sectional area (aₐ) is calculated from a formula
aₐ=Aₘₐₓ*(0.5+tan⁻(P_{term)),}
wherein Pₜₑᵣₘ represents an intermediate pressure term, and arterial compliance, Cₐ, is calculated from a formula
$${C}_{a} = \frac{{A}_{max}}{( ( 1 + {P}_{term}^{2} ) * {π}^{2} )} .$$

59. The method of claim 32, wherein said cardiac output is calculated from a formula
$$CO = \frac{SV ( liters ) * 60 ( sec ) / 1 ( min )}{HeartPeriod ( sec )} .$$

60. The method of claim 32, wherein said electrical measurement of the heart comprises electrical potential of the heart.

61. The method of claim 60, wherein said electrical potential of the heart is measured by an electrocardiogram.

62. The method of claim 32, wherein said electromagnetic radiation absorption is measured on at least one location on the subject's body selected from an arm, hand, finger, ear canal, ear lobe, foot, toe, nare, tongue, back, neck and forehead.

63. The method of claim 32, wherein said electromagnetic radiation absorption is measured on multiple locations on the subject's body selected from an arm, hand, finger, ear canal, ear lobe, foot, toe, nare, tongue, back, neck and forehead.

64. The method of claim 32, wherein said electromagnetic radiation absorption is measured on at least one location on the subject's body selected from the neck and forehead.

65. The method of claim 32, wherein said first and second non-invasive measurements comprise time varying measurements.

66. A system for determining a physiologic characteristic associated with cardiac function in a subject, comprising:
interface means adapted to receive at least one non-invasive measurement representing a physiological parameter of the subject, said non-invasive measurement comprising electromagnetic radiation absorption, and demographic information, said demographic information including information reflecting the subject's physical condition;
means for determining at least one temporal plethysmographic value from said electromagnetic radiation absorption; and
means for determining at least one physiologic characteristic from said temporal plethysmographic value and demographic information by using a predetermined phenomenological model, said model being adapted to provide an estimate of a blood volume-time relationship proximate the heart and compute at least one physiologic characteristic associated with cardiac function based on said estimated blood volume-time relationship.

67. The method of claim 66, wherein said subject's physical condition information is selected from the group consisting of the subject's weight, height, age, gender and body mass index.

68. The method of claim 66, wherein said subject's physical condition information is selected from the group consisting of a disease condition, disease diagnosis and therapy for a disease.

69. The system of claim 66, wherein said physiologic characteristic is selected from the group consisting of heart rate, pulse transit time, wave velocity, central blood flow, peripheral blood flow, perfusion, contractility and vasoconstriction.

70. The system of claim 66, wherein said cardiac function is selected from the group consisting of stroke volume, cardiac output and cardiac index.

71. A system for determining a physiologic characteristic associated with cardiac function in a subject, comprising:
interface means adapted to receive at least first and second non-invasive measurements representing physiological parameters of the subject, said first non-invasive measurement comprising an electrical measurement of the heart and said second non-invasive measurement comprising electromagnetic radiation absorption, and demographic information, said demographic information including information reflecting the subject's physical condition;
means for determining at least one temporal relationship from said electrical measurement of the heart;
means for determining at least one temporal plethysmographic value from said electromagnetic radiation absorption; and
means for determining at least one physiologic characteristic from said temporal relationship, temporal plethysmographic value and demographic information by using a predetermined phenomenological model, said model being adapted to provide an estimate of a blood volume-time relationship proximate the heart and compute at least one physiologic characteristic associated with cardiac function based on said estimated blood volume-time relationship.

72. The method of claim 71, wherein said subject's physical condition information is selected from the group consisting of the subject's weight, height, age, gender and body mass index.

73. The method of claim 71, wherein said subject's physical condition information is selected from the group consisting of a disease condition, disease diagnosis and therapy for a disease.

74. The system of claim 71, wherein said physiologic characteristic is selected from the group consisting of heart rate, pulse transit time, wave velocity, central blood flow, peripheral blood flow, perfusion, contractility and vasoconstriction.

75. The system of claim 71, wherein said cardiac function is selected from the group consisting of stroke volume, cardiac output and cardiac index.

76. A system for determining a physiologic characteristic associated with cardiac function in a subject, comprising:
interface means adapted to receive at least one non-invasive measurement representing a physiological parameter of the subject, said non-invasive measurement comprising electromagnetic radiation absorption;
means for determining at least one temporal plethysmographic value from said electromagnetic radiation absorption;
means for performing non-linear scaling of said temporal plethysmographic value according to a physiological parameter selected from the group consisting of minimum blood pressure, maximum blood pressure and a predetermined blood pressure, said predetermined blood pressure being greater than said minimum blood pressure and less than said maximum blood pressure; and
means for determining a time-varied blood pressure from said scaled temporal plethysmographic value.

77. The system of claim 76, wherein a plurality of said electromagnetic radiation absorption measurements are provided.

78. The method of claim 77, wherein a plurality of temporal plethysmographic values is determined from said plurality of electromagnetic radiation absorption measurements.

79. The method of claim 78, wherein said plurality of temporal plethysmographic values is scaled according to at least one of said physiological parameters.

80. A system for determining a physiologic characteristic associated with cardiac function in a subject, comprising:
interface means adapted to receive at least first and second non-invasive measurements representing physiological parameters of the subject, said first non-invasive measurement comprising an electrical measurement of the heart and said second non-invasive measurement comprising electromagnetic radiation absorption, and demographic information, said demographic information including information reflecting the subject's physical condition;
means for determining at least one temporal relationship from said electrical measurement of the heart;
means for determining at least one temporal plethysmographic value from said electromagnetic radiation absorption;
means for determining the extent of a pressure pulse from said first and second non-invasive measurements;
means for determining heart rate from said first and second non-invasive measurements;
means for determining stroke volume from said temporal arterial pressure, extent of a pressure pulse and demographic information; and
means for determining cardiac output from said stroke volume and said heart rate.

81. A method of detecting hypovolemia in a subject, comprising the step of comparing a change in oxygen saturation as a function of changes in inspiratory oxygen percentage to derive a model based estimate of left ventricular ejection fraction, said comparison being made on a heartbeat-to-heartbeat basis.

82. The method of claim 81, including the step of initially providing said subject with oxygen enriched air, said air being inhaled in one breadth by said subject, whereby said subject's arterial oxygen saturation increases for a first period of time and decreases thereafter for a second period of time.

83. The method of claim 82, wherein a decay parameter is determined from said decrease in said subject's arterial oxygen saturation during said second period of time.

84. The method of claim 83, wherein said ejection fraction is determined by calibrating said decay parameter via echocardiography.
